(19) United States
(12) Patent Application Publication
(10) Pub. No.: US 2023/0092885 A1
(43) Pub. Date: Mar. 23, 2023

(54) TARGETED NANOBUBBLE THERAPY

(71) Applicant: CASE WESTERN RESERVE UNIVERSITY, Cleveland, OH (US)

(72) Inventors: AGATA EXNER, Cleveland, OH (US); JAMES BASILION, Cleveland, OH (US); AMIN JAFARI SOJARHOOD, Cleveland, OH (US); AL DELEON, Cleveland, OH (US); RESHANI PERERA, Cleveland, OH (US); MICHAEL KOLIOS, Cleveland, OH (US)

(21) Appl. No.: 17/910,248

(22) PCT Filed: Mar. 12, 2021
(86) PCT No.: PCT/US2021/022172

§ 371 Date: Sep. 8, 2022

Related U.S. Application Data
(60) Provisional application No. 62/988,832, filed on Mar. 12, 2020.

(51) Int. Cl. A61K 41/00; A61M 37/00; A61K 39/00; A61K 9/51; A61K 47/69; A61P 35/00; A61P 13/08; A61K 47/64

(52) U.S. Cl. CPC A61K 41/0033; A61M 37/0092; A61K 39/001195; A61K 9/5123; A61K 47/6925; A61P 35/00; A61P 13/08; A61K 47/64; A61M 2037/0007

(57) ABSTRACT

A method of inducing cell death in a subject includes administering to the subject a plurality of cell targeted nanobubbles that are internalized by the target cell and insonating nanobubbles internalized into the target cell with ultrasound energy effective to promote inertial cavitation of the internalized nanobubbles and apoptosis and/or necrosis of the target cell.

### RELATED APPLICATION

This application claims priority from U.S. Provisional Application No. 62/988,832, filed Mar. 12, 2020, the subject matter of which is incorporated herein by reference in its entirety.

### GOVERNMENT FUNDING

This invention was made with government support under Grant Nos. 5R01EB025741-02 and 1-R01-EB028144-01A1, awarded by The National Institutes of Health, National Institute of Biomedical Imaging and Bioengineering. The United States government has certain rights to the invention.

### TECHNICAL FIELD

This application relates to diagnostic and therapeutic compositions, and more particularly to nanobubbles for diagnostic, therapeutic, and theranostic applications.

### BACKGROUND

Ultrasound contrast agents (UCA) are small gas-filled bubbles with a stabilizing shell made from a variety of materials, such as polymer, protein, or lipid. Other than the traditional applications of these agents in diagnostic ultrasound imaging, UCA have found relevance in therapeutic applications including targeted gene and drug delivery. These adaptable particles are currently being explored as protective therapeutic carriers and as cavitation nuclei to enhance delivery of their payload by sonoporation. Together these functions improve payload circulation half-life and release profiles as well as tissue selectivity and cell uptake. Regardless of the mode of action, it is advantageous, particularly in cancer therapy, for the bubbles to extravasate from the vasculature and arrive at the cellular target site for the desired effect.

Commercial UCA available today are typically designed to serve only as blood pool agents with diameters of 1-8 μm. Although previous methodologies have been developed to reduce bubble size, most of these strategies involve manipulations of microbubbles post formation, such as gradient separation by gravitational forces or by physical filtration or floatation. While effective for selecting nanosized bubbles, these methods introduce potential for sample contamination, reduce bubble yield and stability, and waste stock materials in addition to being labor intensive. Additionally, the applicability of microbubbles as carriers (e.g., in cancer therapy) has been limited by a large size, which typically confines them to the vasculature.

### SUMMARY

Embodiments described herein relate to a targeted nanobubble therapy (TNT) that can provide a drug-free, low toxicity method of inducing highly selective or targeted cell death in a subject. In some embodiments, the therapy or method can include administering to a subject a plurality of cell targeted nanobubbles. Each of the cell targeted nanobubbles can have a membrane that defines at least one internal void, which includes at least one gas, and a targeting moiety that is linked to an external surface of the membrane. The targeting moiety can bind to a cell surface molecule of a target cell, and the nanobubbles can have a size, diameter, and/or composition that facilitates internalization of the cell targeted nanobubbles by the target cell upon binding of the targeting moiety to the cell surface molecule. Following administration of the cell targeted nanobubbles to the subject, cell targeted nanobubbles internalized into the target cell can be insonated with ultrasound energy effective to promote inertial cavitation of the internalized nanobubbles and apoptosis and/or necrosis of the target cell.

In some embodiment, the cell targeted nanobubbles can have an average diameter of about 50 nm to about 400 nm, and the targeting moiety can include at least one of polypeptides, polynucleotides, small molecules, elemental compounds, antibodies, and antibody fragments.

In other embodiments, the targeted cell can be a cancer cell of the subject and the targeting moiety can bind to a cancer cell surface molecule. The cancer cell surface molecule can be a cancer cell antigen on the surface of a cancer cell. For example, the cancer cell antigen can include at least one of 5T4, α2β1 integrin, AXL receptor tyrosine kinase (AXL), B-cell maturation antigen (BCMA), c-MET (Hepatocyte Growth Factor Receptor), C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, collagen receptor, Cripto protein, CS1, delta-like canonical Notch ligand 3 (DLL3), endothelin receptor type B (EDNRB), ephrin A4 (EFNA4), epidermal growth factor receptor (EGFR), EGFRvIll, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), glycoprotein non-metastatic B (GPNMB), guanylate cyclase 2 C (GUCY2C), human epidermal growth factor receptor 2 (HER2), human epidermal growth factor receptor 3 (HERS), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), protein tyrosine phosphatase mu (PTPmu) solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), or trophoblast cell-surface antigen (TROP-2).

In one example, the targeted cell is a prostate cancer cell, the cell surface molecule is PSMA, and the targeting moiety is a PSMA ligand.

In some embodiments, the membrane can be a lipid membrane. The lipid membrane of the cell targeted nanobubbles can further include at least one of glycerol, propylene glycol, pluronic (poloxamer), alcohols or cholesterols at an amount effective to change the modulus and/or interfacial tension of the nanobubble membrane.

In other embodiments, the lipid membrane includes a mixture of at least two of dipalmitoylphosphatidylcholine (DPPC), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA) or PEG functionalized lipids thereof. For example, the mixture of lipids can include at least about 50% by weight of dibehenoylglycerophosphocoline (DBPC) and less than about 50% by weight of a combination of additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof.

In some embodiments, the gas within the internal void of the cell targeted nanobubbles can include a perfluorocarbon gas, such octafluoropropane (C₃F₈).

In other embodiments, the cell targeted nanobubbles can further include at least one therapeutic agent that is contained within the membrane or conjugated to the membrane of each nanobubble. the therapeutic agent can include, for example, at least one chemotherapeutic agent, anti-proliferative agent, biocidal agent, biostatic agent, or anti-microbial agent.

In some embodiments, insonating the internalized nanobubbles induces death of targeted cell without adversely effecting normal cells and tissues in the subject.

In some embodiments, the insonation can be at a duty cycle of about 1% to about 50%, an ultrasound frequency of about 1 MHz to about 50 MHz (e.g., about 1 MHz to about 10 MHz), an intensity of about 0.1 W/cm² to about 3 W/cm², a pressure amplitude of about 50 kPa to about 1 MPa, and a time of about 1 minute to about 30 minutes.

In other embodiments, the insonation can include two ultrasound pulse sequences with pulses of different pressure amplitudes sent to tissue in which the nanobubbles are internalized by cells. In some embodiments, one pulse can have a pressure amplitude greater than the other pulse. For example, one pulse has a pressure amplitude at least twice the other pulse.

In some embodiments, one pulse can be below the nanobubble pressure threshold for inertial cavitation and be followed by one pulse above the threshold pressure threshold for inertial cavitation. For example, for a nanobubble with a pressure threshold of 200 kPA, the first pulse is at 150 kPA, followed by one at 250 kPa. In another example, for a nanobubble with a pressure threshold of 500 kPa, one pulse is 300 kPA and second is at 600 kPa.

In other embodiments, to induce maximum inertial cavitation, the overall pulse length may also be longer (10-30 cycles) than a typical imaging pulse (3-6 cycles).

In some embodiments, the pulse sequences can be provided from a non-focused transducer, which is distinct from typical focused ultrasound transducers used for drug delivery and ultrasound therapy, such as histotripsy.

In still other embodiments, the method can be used to treat lesions including wide-spread cancer micrometastasis, such as in liver or bone, which cannot be easily visualized and on which focused ultrasound cannot be used.

In still other embodiment, the method and therapy can be used to induce death of prokaryotic cells of microorganisms and treat infections.

Other embodiments described herein relate to a method of treating cancer in a subject in need thereof. The method can include administering to the subject a plurality of cancer cell targeted nanobubbles. Each of the cancer cell targeted nanobubbles can have a membrane that defines at least one internal void, which includes at least one gas, and a targeting moiety that is linked to an external surface of the membrane. The targeting moiety can bind to a cancer cell surface molecule of a target cancer cell. The cancer cell targeted nanobubbles can have a size and/or diameter that facilitates internalization of the nanobubbles by the target cancer cell upon binding of the targeting moiety to the cancer cell surface molecule.

Following administration of the cancer cell targeted nanobubbles to the subject and internalization of the cancer cell targeted nanobubbles into the cancer cells, the internalized nanobubbles can be insonated with ultrasound energy effective to promote inertial cavitation of the internalized nanobubbles and apoptosis and/or necrosis of the target cancer cell.

In some embodiments, the cancer cell surface molecule can be a cancer cell antigen on the surface of a cancer cell. For example, the cancer cell antigen can include at least one of 5T4, α2β1 integrin, AXL receptor tyrosine kinase (AXL), B-cell maturation antigen (BCMA), c-MET (Hepatocyte Growth Factor Receptor), C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, collagen receptor, Cripto protein, CS1, delta-like canonical Notch ligand 3 (DLL3), endothelin receptor type B (EDNRB), ephrin A4 (EFNA4), epidermal growth factor receptor (EGFR), EGFRvIll, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), protein tyrosine phosphatase mu (PTPmu) solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), or trophoblast cell-surface antigen (TROP-2).

In one example, the targeted cancer cell is a prostate cancer cell, the cancer cell surface molecule is PSMA, and the targeting moiety is a PSMA ligand.

Still other embodiments relate to a system for treating cancer in a subject. The system can include an ultrasound source configured to non-invasively deliver ultrasound energy to cancer cells in the subject, a plurality of cancer cell targeted nanobubbles, and a controller coupled to the ultrasound source. Each of the cancer cell targeted nanobubbles can have a membrane that defines at least one internal void, which includes at least one gas, and a targeting moiety that is linked to an external surface of the membrane. The targeting moiety can bind to a cancer cell surface molecule of a target cancer cell. The cancer cell targeted nanobubbles can have a size and/or diameter that facilitates internalization of the nanobubbles by the target cancer cell upon binding of the targeting moiety to the cancer cell surface molecule. The controller coupled to the ultrasound source can be configured to cause insonation of the cancer cells during an insonation time and promote inertial cavitation of nanobubbles internalized by the cancer cells.

In some embodiments, the insonation can be at a duty cycle of about 1% to about 50%, an ultrasound frequency of about 1 MHz to about 50 MHz (e.g., about 1 MHz to about 10 MHz), an intensity of about 0.1 W/cm² to about 3 W/cm², a pressure amplitude of about 50 kPa to about 1 MPa, and a time of about 1 minute to about 30 minutes.

In other embodiments, the insonation can include two ultrasound pulse sequences with pulses of different pressure amplitudes sent to tissue in which the nanobubbles are internalized by cells. In some embodiments, one pulse can have a pressure amplitude greater than the other pulse. For example, one pulse has a pressure amplitude at least twice the other pulse.

In some embodiments, one pulse can be below the nanobubble pressure threshold for inertial cavitation and be followed by one pulse above the threshold pressure threshold for inertial cavitation. For example, for a nanobubble with a pressure threshold of 200 kPA, the first pulse is at 150 kPA, followed by one at 250 kPa. In another example, for a nanobubble with a pressure threshold of 500 kPa, one pulse is 300 kPA and second is at 600 kPa.

In other embodiments, to induce maximum inertial cavitation, the overall pulse length may also be longer (10-30 cycles) than a typical imaging pulse (3-6 cycles).

In some embodiments, the pulse sequences can be provided from a non-focused transducer, which is distinct from typical focused ultrasound transducers used for drug delivery and ultrasound therapy, such as histotripsy.

### BRIEF DESCRIPTION OF THE DRAWINGS

FIGS. 1(A-E) are A) a schematic illustrating the idea of the TNT approach. B) Data showing effect of NBs+US outside of the cell is comparable to that inside the cell at a significantly lower NB dose. C) Feasibility data showing acoustically active retention of PSMA-NBs in prostate cancer cells in vivo and at high resolution in vitro (D, E). NBs inside cells can be deployed to kill the cells with high specificity and minimal collateral damage.

FIG. 2 is a flow diagram illustrating a method in accordance with an embodiment.

FIGS. 3(B-C) illustrates (B) a representative ultrasound contrast images and corresponding enhancement (C) of NBs with stiff and flexible shells showing a significant and rapid increase in signal as the driving pressure is increased. Only the pressures adjacent to the largest signal increase are shown.

FIGS. 4(A-E) illustrate images and plots showing in vivo non-linear contrast scanning A) Time-line showing the bubble injection, non-linear and 3D US application. Representative tumor images showing the bubble distribution at different time in tumor rim and tumor core for B) PSMA-NB. C) Plain NB. D) Lumason microbubble. E) Average signal intensity in tumor rim and core plotted as a function of time for PSMA-NB (i), plain NB (ii), and Lumason MB (iii). Tumors were imaged at 18 MHz, 5 frames/second for 3 min and 1 frames/second for 16 min.

FIGS. 5(A-C) illustrates a schematic and plots showing 3D ultrasound scanning to visualize bubble distribution in whole tumor. A) timeline showing the 3D US scanning points. B) Representative 3D US images of the tumor showing PSMA-NB, NB, and Lumason at the peak. C) Quantification of 3D US signal intensities at peak and the t=25 min after baseline subtraction. N=3, error bars represent mean □ s.d., * P<0.05.

FIGS. 6(A-C) illustrate a schematic and plots showing 3D ultrasound scanning to visualize bubble distribution in whole tumor. A) timeline showing the 3D US scanning points. B) Representative 3D US images of the tumor showing PSMA-NB, NB, and Lumason at the baseline, 25 min post injection, and after cardiac puncture. C) Quantification of 3D US signal intensities before and after cardiac puncture for PSMA-NB, plain Nb, and Lumasonin after baseline subtraction. N=3, error bars represent mean □ s.d., * P<0.05.

FIGS. 7(A-B) illustrate histology images showing the Cy5.5-PSMA-NB accumulation and extravasation in tumor that were excise after cardiac puncture. A) Representative images of tumor tissues showing the PSMA expression (cyan), vasculature (CD31 expression, red), and PSMA-NB or plain NB distribution (green). B) The signal intensities of bubbles, PSMA and vessel are expression as the percentage of total cell fluorescence in tumor section. Cy5.5-PSMA-NB signal in both tumor rim and core was significantly higher from that of NB signal in both tumor rim and core. N=3, error bars represent mean □ s.d., * P<0.001.

FIGS. 8(A-B) illustrate (A) Representative US images of PSMA-positive PC3pip cells incubated with PSMA-NB at different time points after the initial 1 h NB exposure. (B) Acoustic activity of PSMA-NB and NB incubated PC3pip cells and PSMA-negative PC3flu cells at different times post treatment. PSMA-NB incubated PC3pip shows significantly high acoustic activity at t=0 to t=24 h time points compared to all the other groups. n=3, error bars represent mean±s.d., * denotes statistically significant differences (p<0.05) from all other groups at each time point.

FIGS. 9(A-E) illustrate (A) Representative confocal images of PSMA-NB and NB distribution in PC3pip cells; 100× (blue-nuclei, red-NB, and green-late endosome/lysosomes). Zoomed merged images of (B) PSMA-NB and (C) plain NB incubated PC3pip cells. (D) Representative confocal images of PSMA-NB distribution in PC3pip cells after 24 h exposure (blue-nuclei, red-NB, and green-endosome). Zoomed merged images of (E) PSMA-NB and (F) plain NB incubated PC3pip cells. PSMA-NB shows high co-localization in late endosomal/lysosomal vesicles (yellow).

FIGS. 10(A-C) illustrate plots showing (A) Head-space GC/MS analysis of C₃F₈ gas generated by NB; eluting at 3.37 min (B) Calibration curve for various concentrations of bubbles vs peak area correspond to C₃F₈ gas (C) Head space GC/MS analysis of C₃F₈ gas generated by PSMA-NB and plain NB internalized PC3pip cell suspension.

FIGS. 11(A-B) illustrate (A) Representative US images of subcutaneous tissue obtained after injection of labeled cells (PSMA-NB incubated cells) and unlabeled cells at different time points at 0.1 MI value. (B) Average US signal intensities at each time points.

FIG. 11 illustrates tumor images showing the bubble distribution at different time in tumor rim and tumor core for PSMA-NB for other replicates.

FIG. 12 is a schematic diagram of tumor model and PSMA-targeted NBs and non-targeted NBs.

FIGS. 13(A-C) illustrate PSMA-targeted NBs provide greater tumor enhancement compared to LUMASON. (A) Representative ultrasonographic images of PC3pip orthotopic tumor and liver after injection of PSMA targeted NBs and clinically available MB (LUMASON). The first and second rows showed the B-mode and CHI mode images of tumor and liver before UCAs injection. The third to the fifth rows showed the CHI images at different time points after UCAs administration. The imaging intensity in the tumor and liver from mice received PSMA-targeted NBs was apparently higher than those in animals received LUMASON at different time points. Scale bar is 0.5 cm. (B1) The time intensity curves (TIC) of the PC3pip orthotopic tumor after i.v. administration of PSMA-targeted NBs (n=11) and LUMASON (n=3). (B2) The time intensity curve (TIC) of the liver after i.v. administration of PSMA-targeted NBs (n=4) and LUMASON (n=3). (C) Comparison of the UCA kinetic parameters between PSMA-targeted NBs and LUMASON in the tumors or livers. Data as mean±standard deviation; *p<0.05, PSMA-targeted NBs group vs. LUMASON group.

FIGS. 14(A-C) illustrate images and plots showing PSMA-targeted NBs provide greater tumor enhancement as compared to non-targeted NBs. (A) Representative ultrasonographic images of PC3pip orthotopic tumor after injection of PSMA-targeted NBs and non-targeted NBs (n=11). The first and second columns showed the B-mode and CHI mode images of tumor before UCAs injection, respectively. The third to the fifth columns showed the CHI images at different time points after UCAs administration. Scale bar is 0.5 cm. (B1) The time intensity curves (TIC) of the PC3pip orthotopic tumor after i.v. administration of PSMA-targeted NBs and non-targeted NBs. (B2) US signal obtained from non-targeted NBs measurements were used to normalize the signal from PSMA-targeted NBs. The normalized signal enhancement means (Intensity_{PSMA-targeted NBs}−Intensity_{non-targeted NBs}) (C) Comparison of A-targeted NBs and non-targeted NBs in tumor. Data are presented by deviation (n=11); *p<0.05 targeted NB vs. non-targeted NBs.

FIGS. 15(A-B) illustrate plots showing PSMA-targeted NBs and non-targeted NBs provide more tumor enhancement in small tumors (Group A) as compared to that in large tumors (Group B). (A) The time intensity curves (TIC) of tumor aft i.v. administration of PSMA-targeted NBs and non-targeted NBs in Group A (n=7) and Group B (n=4). (B) tic parameters between Group A (n=7) and Group B (n=4). Data are presented as standard deviation; *p<0.05, group A vs. group B.

FIGS. 16(A-B) illustrate images and plots showing PSMA-targeted NBs enable prolonged imaging and greater US signal in PSMA-positive PC3pip tumors after removing nanobubbles from the circulation. (A) The first row showed the B-mode image of the tumor and liver before injection. The second row showed the CHI of the tumor and liver before bubble burst. The third row showed the CHI of the tumor and liver after bubble burst. Scale bar is 0.5 cm. (B) The average signal intensities of bubbles in the tumor and liver before and after the burst. Data are represented as mean±standard deviation, *p<0.05, targeted group vs. non-targeted group, n=4.

FIGS. 17(A-B) illustrate histological images of Cy5.5 and CD31 signal in tumors treated with PSMA-targeted NBs or non-targeted NBs after perfusion. (magnification:20×) (A) Cy5.5 and CD31 signals in the tumor after perfusion. N=3 for both PSMA-1-targeted group and non-targeted group. (B1) Quantification of fluorescence ratio (total bubbles fluorescence/vessels fluorescence per field). Data are presented as mean±standard deviation; *p<0.05, targeted group vs. non-targeted group, n=3. (B2) Quantification of fluorescence ratio (total bubbles fluorescence/cells fluorescence per field).

FIG. 18 illustrates a schematic of a procedure for administering and insonating PSMA-NBs to mice.

FIG. 19 illustrates images of PSMA positive tumors and PSMA negative tumors treated with the PSMA-NBs and US.

FIG. 20 illustrates images of PSMA positive tumors and PSMA negative tumors treated with US only (no PSMA-NBs).

FIG. 21 illustrates images showing PSMA positive tumors treated with PSMA-NBs and US.

FIG. 22 illustrates images showing PSMA negative tumors treated with PSMA-NBs and US.

### DETAILED DESCRIPTION

All scientific and technical terms used in this application have meanings commonly used in the art unless otherwise specified. The definitions provided herein are to facilitate understanding of certain terms used frequently herein and are not meant to limit the scope of the application.

As used herein and in the appended claims, the singular forms “a”, “an”, and “the” include plural references unless the context clearly dictates otherwise. Furthermore, the terms “a” (or “an”), “one or more” and “at least one” can be used interchangeably herein. The terms “comprising”, “including”, “having” and “constructed from” can also be used interchangeably.

The term “stable cavitation” refers to gas voids of nanobubbles that have a tendency to increase in size and vibrate without imploding. The gas voids vibrate when exposed to a pressure field but do not implode. In stable cavitation, a collection of gas voids of nanobubbles tend to operate in a relatively stable manner as long as a pressure field capable of producing rectified diffusion exists.

The term “inertial cavitation” refers to the oscillation and violent collapse of gas voids of nanobubbles induced by an applied pressure field, usually at the gas voids' resonance frequency. When the gas void or nanobubble implode in a cell, they exert a concentrated, high pressure force against the cell, which can destroy cell organelles, cytoskeleton, and denature proteins in the cell. In addition to causing cell damage, inertial cavitation may also generate free radicals.

The term “neoplastic disorder” can refer to a disease state in a subject in which there are cells and/or tissues which proliferate abnormally. Neoplastic disorders can include, but are not limited to, cancers, sarcomas, tumors, leukemias, lymphomas, and the like.

The term “neoplastic cell” can refer to a cell that shows aberrant cell growth, such as increased, uncontrolled cell growth. A neoplastic cell can be a hyperplastic cell, a cell from a cell line that shows a lack of contact inhibition when grown in vitro, a tumor cell, or a cancer cell that is capable of metastasis in vivo. Alternatively, a neoplastic cell can be termed a “cancer cell.” Non-limiting examples of cancer cells can include melanoma, breast cancer, ovarian cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, thymoma, lymphoma cells, melanoma cells, sarcoma cells, leukemia cells, retinoblastoma cells, hepatoma cells, myeloma cells, glioma cells, mesothelioma cells, and carcinoma cells.

The term “tumor” can refer to an abnormal mass or population of cells that result from excessive cell division, whether malignant or benign, and all pre-cancerous and cancerous cells and tissues.

The terms “treating” or “treatment” of a disease (e.g., a neoplastic disorder) can refer to executing a treatment protocol to eradicate at least one neoplastic cell. Thus, “treating” or “treatment” does not require complete eradication of neoplastic cells.

The term “polymer” can refer to a molecule formed by the chemical union of two or more chemical units. The chemical units may be linked together by covalent linkages. The two or more combining units in a polymer can be all the same, in which case the polymer may be referred to as a homopolymer. The chemical units can also be different and, thus, a polymer may be a combination of the different units. Such polymers may be referred to as copolymers.

The term “subject” can refer to any animal, including, but not limited to, humans and non-human animals (e.g., rodents, arthropods, insects, fish (e.g., zebrafish)), non-human primates, ovines, bovines, ruminants, lagomorphs, porcines, caprines, equines, canines, felines, ayes, etc.), which is to be the recipient of a particular treatment.

Embodiments described herein relate to a targeted nanobubble therapy (TNT) that can provide a drug-free, low toxicity approach of inducing highly selective or targeted cell death in a subject. The TNT can use nanobubbles (NB) that are targeted to cell surface molecules of the targeted cells and are internalized into the targeted cells. Once inside or internalized into the target cells, the cell targeted NBs can be insonated to promote inertial cavitation and/or destruction of the internalized NBs using a nanobubble-specific ultrasound pulse. The unique combination of NBs interacting with ultrasound inside the targeted cell can lead to the highly selective or targeted cell death. The TNT capitalizes on the specific targeting of nanobubbles only to those cells that express the specific cell surface molecule—and then triggering their inertial cavitation with ultrasound.

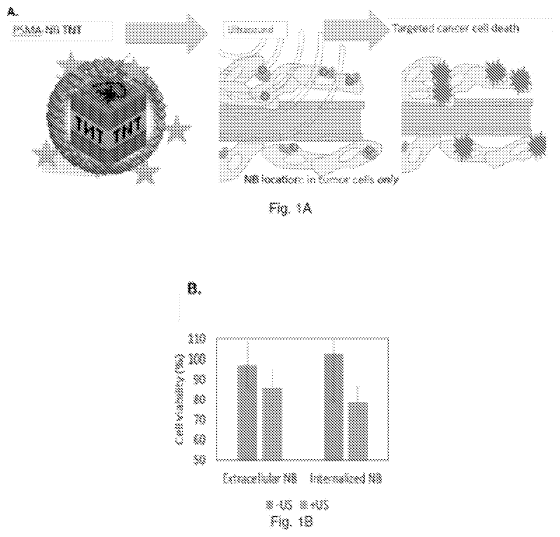

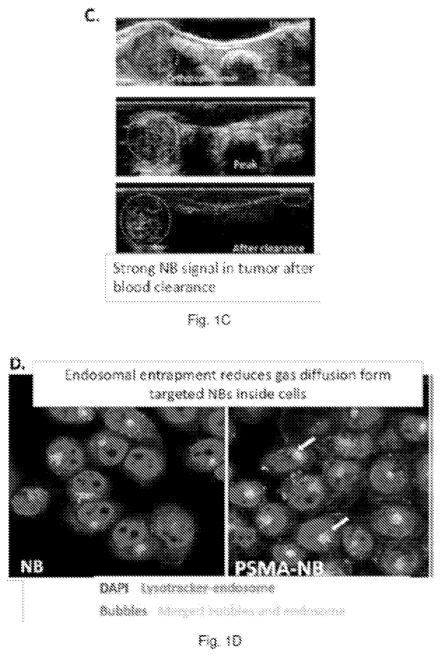

By way of example, NBs can be targeted to PSMA via a highly selective ligand. Since PSMA levels are reported to be associated with the aggressiveness of prostate cancer (PCa), PSMA targeting NBs would be highly selective to PSMA-expressing, or aggressive, tumors (FIG. 1). Once the PSMA targeted NBs are internalized by the PCa cells and the remaining NB s (which alone have zero toxicity) clear out of the blood stream, a series of ultrasound pulses can be used to inertially cavitate or burst the NBs inside the cancer cells. This results in highly focused treatment of cancer cells leaving normal cells untouched. As ultrasound is frequently utilized in many cancer diagnosis and biopsy procedures, the same equipment and workflow can be applied by doctors already familiar with the techniques, thus lowering costs and expediting clinical translation.

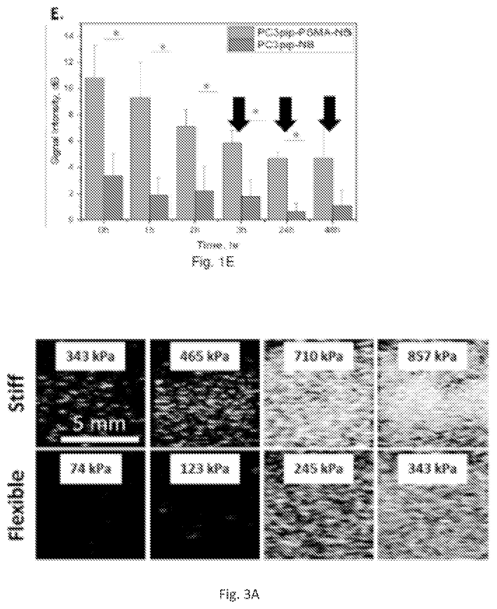

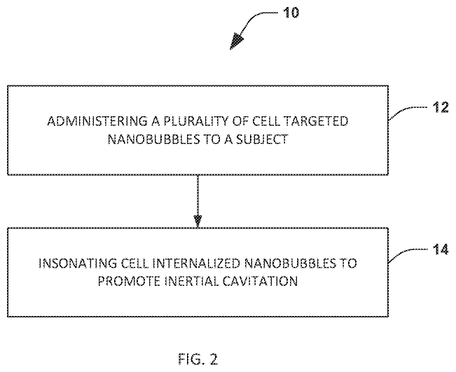

FIG. 2 is a flow chart illustrating a therapy or method 10 of inducing death in accordance with an embodiment described herein. In the therapy or method 10 of inducing cell death, such as cancer cell death or microorganism cell death, at step 12, a plurality of cell targeted nanobubbles, which can be internalized by a target can be administered to a subject.

Each of the cell targeted nanobubbles can have a membrane, such as a lipid membrane, that defines at least one internal void, which includes at least one gas, and a targeting moiety that is linked to an external surface of the lipid membrane. The target moiety can bind to a cell surface molecule of a target cell and the cell targeted nanobubble upon binding of the targeting moiety to the cell surface molecule can be internalized by the target cell.

The lipid membrane can exhibit selective activation and/or cavitation to known ultrasound pressures. In some embodiments, the lipid membrane can be specifically modified to elicit cavitation and nanobubble collapse at predictable pressures. This can avoid collateral damage and activation of other nanoscale gas nucleation sites. The composition of the lipid membrane used to form the cell targeted nanobubbles also enables the cavitation threshold to be significantly lowered.

In some embodiments, the lipid membrane can include, for example, a plurality of lipids, an edge-activator, which is incorporated between lipids of the membrane and enhances the flexibility of the nanobubbles, a membrane stiffener, which is incorporated on an outer surface of the membrane and enhances the membranes resistance to tearing, and, other additives, such as pluronic (poloxamer), alcohols and cholesterols, that change the modulus and/or interfacial tension of the bubble shell.

In other embodiments, each of the nanobubbles can include a hydrophilic outer domain at least partially defined by hydrophilic heads of the lipid and a hydrophobic inner domain at least partially defined by hydrophobic tails of the lipid. An edge activator, such as propylene glycol, can at least partially extend between the lipids from the outer domain to the inner domain. The glycerol can be provided on the outer domain of the nanobubbles and extend partially between hydrophilic heads of the lipids. The gas, which is encapsulated by the membrane, can have a low solubility in water (e.g., hydrophobic gas) and include, for example, a perfluorocarbon, such as perfluoropropane or perfluorobutane, sulfur hexafluoride, carbon dioxide, nitrogen (N₂), oxygen (O₂), and air.

In some embodiments, each of the cell targeted nanobubbles can have a size that facilitates extravasation of the cell targeted nanobubbles and internalization of the cell targeted nanobubbles by the target cell upon binding of the targeting moiety to the cell surface molecule. For example, each of the nanobubbles can have a size (diameter) of about 30 nm to about 600 nm or about 100 nm to about 500 nm (e.g., about 300 nm), depending upon the particular lipids, edge activator, and membrane stiffener as well as the method used to form the nanobubble (described in greater detail below).

The cell targeted nanobubbles can have a lipid concentration that enhances the in vivo circulation stability of the nanobubbles. It was found that a higher lipid concentration correlated with an increase in stability and longer circulation of the nanobubbles upon administration to a subject. In some embodiments, the cell targeted nanobubbles can have a lipid concentration of at least about 2 mg/ml, at least about 3 mg/ml, at least about 4 mg/ml, at least about 5 mg/ml, about 6 mg/ml, at least about 7 mg/ml, at least about 8 mg/ml, at least about 9 mg/ml, at least about 10 mg/ml, at least about 11 mg/ml, at least about 12 mg/ml or more. In other embodiments, the lipid concentration of the cell targeted nanobubbles can be about 5 mg/ml to about 12 mg/ml, about 6 mg/ml to about 12 mg/ml, about 7 mg/ml to about 12 mg/ml, about 8 mg/ml to about 12 mg/ml, about 9 mg/ml to about 12 mg/ml, about 10 mg/ml to about 12 mg/ml, or at least about 10 mg/ml.

The plurality of lipids comprising the membrane or shell can include any naturally-occurring, synthetic or semi-synthetic (i.e., modified natural) moiety that is generally amphipathic or amphiphilic (i.e., including a hydrophilic component and a hydrophobic component). Examples of lipids, any one or combination of which may be used to form the membrane, can include: phosphocholines, such as 1-alkyl-2-acetoyl-sn-glycero 3-phosphocholines, and 1-alkyl-2-hydroxy-sn-glycero 3-phosphocholines; phosphatidylcholine with both saturated and unsaturated lipids, including dioleoylphosphatidylcholine, dimyristoylphosphatidylcholine, dipentadecanoylphosphatidylcholine, dilauroylphosphatidylcholine, dipalmitoylphosphatidylcholine (DPPC), dibehenoylglycerophosphocoline (DBPC), distearoylphosphatidylcholine (DSPC), and diarachidonylphosphatidylcholine (DAPC); phosphatidylethanolamines, such as dioleoylphosphatidylethanolamine, dipalmitoylphosphatidylethanolamine (DPPE), and distearoylphosphatidylethanolamine (DSPE); phosphatidylserine; phosphatidylglycerols, including distearoylphosphatidylglycerol (DSPG); phosphatidylinositol; sphingolipids, such as sphingomyelin; glycolipids, such as ganglioside GM1 and GM2; glucolipids; sulfatides; glycosphingolipids; phosphatidic acids, such as dipalmitoylphosphatidic acid (DPPA) and distearoylphosphatidic acid (DSPA); palmitic acid; stearic acid; arachidonic acid; oleic acid; lipids bearing polymers, such as chitin, hyaluronic acid, polyvinylpyrrolidone or polyethylene glycol (PEG); lipids bearing sulfonated mono-, di-, oligo- or polysaccharides; cholesterol, cholesterol sulfate, and cholesterol hemisuccinate; tocopherol hemisuccinate; lipids with ether and ester-linked fatty acids; polymerized lipids (a wide variety of which are well known in the art); diacetyl phosphate; dicetyl phosphate; stearylaamine; cardiolipin; phospholipids with short chain fatty acids of about 6 to about 8 carbons in length; phospholipids with medium chain fatty acids of about 10 to about 16 carbons in length; phospholipids with long chain fatty acids of about 18 to about 24 carbons in length; synthetic phospholipids with asymmetric acyl chains, such as, for example, one acyl chain of about 6 carbons and another acyl chain of about 12 carbons; ceramides; non-ionic liposomes including niosomes, such as polyoxyalkylene (e.g., polyoxyethylene) fatty acid esters, polyoxyalkylene (e.g., polyoxyethylene) fatty alcohols, polyoxyalkylene (e.g., polyoxyethylene) fatty alcohol ethers, polyoxyalkylene (e.g., polyoxyethylene) sorbitan fatty acid esters (such as, for example, the class of compounds referred to as TWEEN (commercially available from ICI Americas, Inc., Wilmington, Del.), glycerol polyethylene glycol oxystearate, glycerol polyethylene glycol ricinoleate, alkyloxylated (e.g., ethoxylated) soybean sterols, alkyloxylated (e.g., ethoxylated) castor oil, polyoxyethylene-polyoxypropylene polymers, and polyoxyalkylene (e.g., polyoxyethylene) fatty acid stearates; sterol aliphatic acid esters including cholesterol sulfate, cholesterol butyrate, cholesterol isobutyrate, cholesterol palmitate, cholesterol stearate, lanosterol acetate, ergosterol palmitate, and phytosterol n-butyrate; sterol esters of sugar acids including cholesterol glucuronide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; long chain alcohols including n-decyl alcohol, lauryl alcohol, myristyl alcohol, cetyl alcohol, and n-octadecyl alcohol; 6-(5-cholesten-3β-yloxy)-1-thio-β-D-galactopyranoside; digalactosyldiglyceride; 6-(5-cholesten-3β-yloxy)hexyl-6-amino-6-deoxy-1-thio-β-D-galactopyranoside; 6-(5-cholesten-3β-yloxy)hexyl-6-amino-6-deoxyl-1-thio-α-D-mannopyranoside; 12-(((7′-diethylaminocoumarin-3-yl)carbonyl)methylamino)octadecanoic acid; N-[12-(((7′-diethylaminocoumarin-3-yl)carbonyl)methylamino)octadecanoyl]-2-aminopalmitic acid; cholesteryl(4′-trimethylammonio)butanoate; N-succinyldioleoylphosphatidylethanolamine; 1,2-dioleoyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine and palmitoylhomocysteine; and/or any combinations thereof.

In some embodiments, the plurality of lipids used to form the membrane can include a mixture of phospholipids having varying acyl chain lengths. For example, the lipids can include a mixture of at least two of dipalmitoylphosphatidylcholine (DPPC), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or PEG functionalized lipids thereof.

In other embodiments, the mixture of phospholipids having varying acyl chain length can include dibehenoylglycerophosphocoline (DBPC) and one or more additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof.

In some embodiments, the mixture of phospholipids can include at least about 40%, at least about 50%, at least about 60%, at least about 70%, or at about 80%, by weight of dibehenoylglycerophosphocoline (DBPC); and less than about 60%, less than about 50%, less than about 40%, less than about 30%, or less than about 20%, by weight, of a combination of additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof. The PEG can have a molecular weight of about 1000 to about 5000 Da, for example, about 2000 Da.

In some embodiments, the mixture of phospholipids can include about 40% to about 80%, about 50% to about 70%, or about 55% to about 65% (e.g., about 60%) by weight dibehenoylglycerophosphocoline (DBPC); and about 20% to about 60%, about 30% to about 50%, or about 35% to about 45% (e.g., about 40%) by weight of a combination of additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof.

In other embodiments, the one or more additional phospholipids can include, consist essentially of, or consists of a combination of dipalmitoylphosphatidic acid (DPPA), dipalmitoylphosphatidylethanolamine (DPPE), and PEG functionalized distearoylphosphatidylethanolamine (DSPE)

In still other embodiments, the mixture of phospholipids can include dibehenoylglycerophosphocoline (DBPC), dip almitoylphosphatidic acid (DPPA), dipalmitoylphosphatidylethanolamine (DPPE), and PEG functionalized distearoylphosphatidylethanolamine (DSPE) at a ratio of, for example, about 6:1:1:1 by weight.

In some embodiments, the edge-activator, which is incorporated between lipids of the membrane of each nanobubble and enhances the flexibility of the nanobubbles can include a co-surfactant, such as propylene glycol, which enhances the effectiveness of phospholipid surfactants. The edge activator can be provided in each of the nanobubbles at an amount effective to cause separation of lipid domains of the nanobubble and form defects that absorb excessive pressure, which could have caused lipid “domain” tearing. Other edge activators, which can be substituted for propylene glycol or used in combination with propylene glycol, can include cholesterol, sodium cholate, limonene, oleic acid, and/or span 80.

In some embodiments, the amount of propylene glycol provided in the nanobubbles can be about 0.05 ml to about 0.5 ml, about 0.06 ml to about 0.4 ml, about 0.07 ml to about 0.3 ml, about 0.08 ml to about 0.2 ml, or about 0.1 ml, per 1 ml of hydrated lipids.

In other embodiments, a membrane stiffener, which is incorporated on the outer surface of the membrane of each nanobubble and enhances the membranes resistance to tearing, includes glycerol. Glycerol can be provided on the membrane of each of the nanobubbles at an amount effective to stiffen the membrane and improve the membrane's resistance to lipid “domain” tearing. The amount of glycerol provided on the membranes of the nanobubbles can be about 0.05 ml to about 0.5 ml, about 0.06 ml to about 0.4 ml, about 0.07 ml to about 0.3 ml, about 0.08 ml to about 0.2 ml, or about 0.1 ml, per 1 ml of hydrated lipids.

The membranes defining the nanobubbles can be concentric or otherwise and have a unilamellar configuration (i.e., comprised of one monolayer or bilayer), an oligolamellar configuration (i.e., comprised of about two or about three monolayers or bilayers), or a multilamellar configuration (i.e., comprised of more than about three monolayers or bilayers). The membrane can be substantially solid (uniform), porous, or semi-porous.

The internal void space defined by the membrane can include at least one gas. The gas can have a low solubility in water and be, for example, a perfluorocarbon, such as perfluoropropane (e.g., octafluoropropane) or perfluorobutane. The internal void can also include other gases, such as carbon dioxide, sulfur hexafluoride, air, nitrogen (N2), oxygen (O2), and helium.

In some embodiments, the nanobubbles can include a linker to link a targeting moiety and, optionally, a therapeutic agent to the membrane of each nanobubble. The linker can be of any suitable length and contain any suitable number of atoms and/or subunits. The linker can include one or combination of chemical and/or biological moieties. Examples of chemical moieties can include alkyl groups, methylene carbon chains, ether, polyether, alkyl amide linkers, alkenyl chains, alkynyl chains, disulfide groups, and polymers, such as poly(ethylene glycol) (PEG), functionalized PEG, PEG-chelant polymers, dendritic polymers, and combinations thereof. Examples of biological moieties can include peptides, modified peptides, streptavidin-biotin or avidin-biotin, polyaminoacids (e.g., polylysine), polysaccharides, glycosaminoglycans, oligonucleotides, phospholipid derivatives, and combinations thereof.

The cell targeted nanobubbles can also include other materials, such as liquids, oils, bioactive agents, diagnostic agents, therapeutic agents, photoacoustic agents (e.g., sudan black), and/or nanoparticles (e.g., iron oxide). The materials can be encapsulated by the membrane and/or linked or conjugated to the membrane.

The targeting moiety binds to a cell surface molecule of a target cell and/or tissue and is capable of targeting and/or adhering the nanobubble to the targeted cell and/or tissue of interest. In some embodiments, the targeting moiety can comprise any molecule, or complex of molecules, which is/are capable of interacting with a cell surface or extracellular molecule or biomarker of the cell. The cell surface molecule can include, for example, a cellular protease, a kinase, a protein, a cell surface receptor, a lipid, and/or fatty acid.

In certain embodiments, the targeting moiety specifically binds the cell surface molecule of the target cell. As used herein, a first molecule “specifically binds” to a second molecule if it binds to or associates with the second molecule with an affinity or Ka (that is, an equilibrium association constant of a particular binding interaction with units of 1/M) of, for example, greater than or equal to about 10⁵ M⁻¹. In certain embodiments, the first molecule binds to the second molecule with a Ka greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (KD) of a particular binding interaction with units of M (e.g., 10⁻⁵ M to 10⁻¹³ M, or less). In certain aspects, specific binding means binding to the target molecule with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the first molecule for the target can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

In some embodiments, the targeting moiety can include, but is not limited to, synthetic compounds, natural compounds or products, macromolecular entities, bioengineered molecules (e.g., polypeptides, lipids, polynucleotides, antibodies, antibody fragments), and small entities (e g, small molecules, neurotransmitters, substrates, ligands, hormones and elemental compounds).

In one example, the targeting moiety can comprise an antibody, such as a monoclonal antibody, a polyclonal antibody, or a humanized antibody, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent targeting moieties including without limitation: monospecific or bispecific antibodies, such as disulfide Fv fragments, scFv tandems ((scFv)2 fragments), diabodies, tribodies or tetrabodies, which typically are covalently linked or otherwise stabilized (i.e., leucine zipper or helix stabilized) scFv fragments; and receptor molecules, which naturally interact with a desired target molecule.

Preparation of antibodies may be accomplished by any number of well-known methods for generating antibodies. These methods typically include the step of immunization of animals, typically mice, with a desired immunogen (e.g., a desired target molecule or fragment thereof). Once the mice have been immunized and boosted one or more times with the desired immunogen(s), antibody-producing hybridomas may be prepared and screened according to well-known methods. See, for example, Kuby, Janis, Immunology, Third Edition, pp. 131-139, W.H. Freeman & Co. (1997), for a general overview of monoclonal antibody production, that portion of which is incorporated herein by reference.

The targeting moiety need not originate from a biological source. The targeting moiety may, for example, be screened from a combinatorial library of synthetic peptides. One such method is described in U.S. Pat. No. 5,948,635, incorporated herein by reference, which describes the production of phagemid libraries having random amino acid insertions in the pIII gene of M13. This phage may be clonally amplified by affinity selection.

The immunogens used to prepare targeting moieties having a desired specificity will generally be the target molecule, or a fragment or derivative thereof. Such immunogens may be isolated from a source where they are naturally occurring or may be synthesized using methods known in the art. For example, peptide chains may be synthesized by 1-ethyl-3-[dimethylaminoproply]carbodiimide (EDC)-catalyzed condensation of amine and carboxyl groups. In certain embodiments, the immunogen may be linked to a carrier bead or protein. For example, the carrier may be a functionalized bead such as SASRIN resin commercially available from Bachem, King of Prussia, Pa. or a protein such as keyhole limpet hemocyanin (KLH) or bovine serum albumin (BSA). The immunogen may be attached directly to the carrier or may be associated with the carrier via a linker, such as a non-immunogenic synthetic linker (for example, a polyethylene glycol (PEG) residue, amino caproic acid or derivatives thereof) or a random, or semi-random polypeptide.

In certain embodiments, it may be desirable to mutate a binding region of the polypeptide targeting moiety and select for a targeting moiety with superior binding characteristics as compared to the un-mutated targeting moiety. This may be accomplished by any standard mutagenesis technique, such as by PCR with Taq polymerase under conditions that cause errors. In such a case, the PCR primers could be used to amplify scFv-encoding sequences of phagemid plasmids under conditions that would cause mutations. The PCR product may then be cloned into a phagemid vector and screened for the desired specificity, as described above.

In other embodiments, the targeting moiety may be modified to make them more resistant to cleavage by proteases. For example, the stability of a targeting moiety comprising a polypeptide may be increased by substituting one or more of the naturally occurring amino acids in the (L) configuration with D-amino acids. In various embodiments, at least 1%, 5%, 10%, 20%, 50%, 80%, 90% or 100% of the amino acid residues of targeting moiety may be of the D configuration. The switch from L to D amino acids neutralizes the digestion capabilities of many of the ubiquitous peptidases found in the digestive tract. Alternatively, enhanced stability of a targeting moiety comprising a peptide bond may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of a targeting moiety may be achieved by intercalating one or more dextrorotatory amino acids (such as, dextrorotatory phenylalanine or dextrorotatory tryptophan) between the amino acids of targeting moiety. In exemplary embodiments, such modifications increase the protease resistance of a targeting moiety without affecting the activity or specificity of the interaction with a desired target molecule.

In certain embodiments, antibodies or variants thereof may be modified to make them less immunogenic when administered to a subject. For example, if the subject is human, the antibody may be “humanized”; where the complimentarily determining region(s) of the hybridoma-derived antibody has been transplanted into a human monoclonal antibody, for example as described in Jones, P. et al. (1986), Nature, 321, 522-525 or Tempest et al. (1991), Biotechnology, 9, 266-273. Also, transgenic mice, or other mammals, may be used to express humanized antibodies. Such humanization may be partial or complete.

In certain embodiments, a targeting moiety as described herein may comprise a homing peptide, which selectively directs the nanobubble to a targeted cell. Homing peptides for a targeted cell can be identified using various methods well known in the art. Many laboratories have identified the homing peptides that are selective for cells of the vasculature of brain, kidney, lung, skin, pancreas, intestine, uterus, adrenal gland, retina, muscle, prostate, or tumors. See, for example, Samoylova et al., 1999, Muscle Nerve, 22:460; Pasqualini et al., 1996 Nature, 380:364; Koivunen et al., 1995, Biotechnology, 13:265; Pasqualini et al., 1995, J. Cell Biol., 130:1189; Pasqualini et al., 1996, Mole. Psych., 1:421, 423; Rajotte et al., 1998, J. Clin. Invest., 102:430; Rajotte et al., 1999, J. Biol. Chem., 274:11593. See, also, U.S. Pat. Nos. 5,622,6999; 6,068,829; 6,174,687; 6,180,084; 6,232,287; 6,296,832; 6,303,573; and 6,306,365.

Phage display technology provides a means for expressing a diverse population of random or selectively randomized peptides. Various methods of phage display and methods for producing diverse populations of peptides are well known in the art. For example, methods for preparing diverse populations of binding domains on the surface of a phage have been described in U.S. Pat. No. 5,223,409. In particular, phage vectors useful for producing a phage display library as well as methods for selecting potential binding domains and producing randomly or selectively mutated binding domains are also provided in U.S. Pat. No. 5,223,409. Similarly, methods of producing phage peptide display libraries, including vectors and methods of diversifying the population of peptides that are expressed, are also described in Smith et al., 1993, Meth. Enzymol., 217:228-257, Scott et al., Science, 249:386-390, and two PCT publications WO 91/07141 and WO 91/07149. Phage display technology can be particularly powerful when used, for example, with a codon based mutagenesis method, which can be used to produce random peptides or randomly or desirably biased peptides (see, e.g., U.S. Pat. No. 5,264,563). These or other well-known methods can be used to produce a phage display library, which can be subjected to the in vivo phage display method in order to identify a peptide that homes to one or a few selected tissues.

In vitro screening of phage libraries has previously been used to identify peptides that bind to antibodies or cell surface receptors (see, e.g., Smith, et al., 1993, Meth. Enzymol., 217:228-257). For example, in vitro screening of phage peptide display libraries has been used to identify novel peptides that specifically bind to integrin adhesion receptors (see, e.g., Koivunen et al., 1994, J. Cell Biol. 124:373-380), and to the human urokinase receptor (Goodson, et al., 1994, Proc. Natl. Acad. Sci., USA 91:7129-7133).

In certain embodiments, the targeting moiety may comprise a receptor molecule, including, for example, receptors, which naturally recognize a specific desired molecule of a target cell. Such receptor molecules include receptors that have been modified to increase their specificity of interaction with a target molecule, receptors that have been modified to interact with a desired target molecule not naturally recognized by the receptor, and fragments of such receptors (see, e.g., Skerra, 2000, J. Molecular Recognition, 13:167-187). A preferred receptor is a chemokine receptor. Exemplary chemokine receptors have been described in, for example, Lapidot et al, 2002, Exp Hematol, 30:973-81 and Onuffer et al, 2002, Trends Pharmacol Sci, 23:459-67.

In other embodiments, the targeting moiety may comprise a ligand molecule, including, for example, ligands which naturally recognize a specific desired receptor of a target cell. Such ligand molecules include ligands that have been modified to increase their specificity of interaction with a target receptor, ligands that have been modified to interact with a desired receptor not naturally recognized by the ligand, and fragments of such ligands.

In still other embodiments, the targeting moiety may comprise an aptamer. Aptamers are oligonucleotides that are selected to bind specifically to a desired molecular structure of the target cell. Aptamers typically are the products of an affinity selection process similar to the affinity selection of phage display (also known as in vitro molecular evolution). The process involves performing several tandem iterations of affinity separation, e.g., using a solid support to which the diseased immunogen is bound, followed by polymerase chain reaction (PCR) to amplify nucleic acids that bound to the immunogens. Each round of affinity separation thus enriches the nucleic acid population for molecules that successfully bind the desired immunogen. In this manner, a random pool of nucleic acids may be “educated” to yield aptamers that specifically bind target molecules. Aptamers typically are RNA, but may be DNA or analogs or derivatives thereof, such as, without limitation, peptide nucleic acids (PNAs) and phosphorothioate nucleic acids.

In yet other embodiments, the targeting moiety may be a peptidomimetic. By employing, for example, scanning mutagenesis to map the amino acid residues of a protein, which is involved in binding other proteins, peptidomimetic compounds can be generated that mimic those residues, which facilitate the interaction. Such mimetics may then be used as a targeting moiety to deliver the nanobubble to a target cell. For instance, non-hydrolyzable peptide analogs of such resides can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al., 1986, J Med Chem 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., 1985, Tetrahedron Lett 26:647; and Sato et al., 1986, J Chem Soc Perkin Trans 1:1231), and β-aminoalcohols (Gordon et al., 1985, Biochem Biophys Res Cummun 126:419; and Dann et al., 1986, Biochem Biophys Res Commun 134:71).

In some embodiments, the targeting moiety binds to an antigen on a target cells. Target cells of interest include, but are not limited to, cells that are relevant to a particular disease or condition, where it is desirable to induce cell death. According to some embodiments, the target cell can be a cancer cell, an immune cell, an endothelial cell, or a prokaryotic cell of a microorganism.

As such, in some embodiments, the target cells are cancer cells. By “cancer cell” it is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a primary tumor, a metastatic tumor, and the like. In certain aspects, the cancer cell is a carcinoma cell.

In some embodiments, the cancer cell antigen can include at least one of 5T4, α2β1 integrin, AXL receptor tyrosine kinase (AXL), B-cell maturation antigen (BCMA), c-MET (Hepatocyte Growth Factor Receptor), C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, collagen receptor, Cripto protein, CS1, delta-like canonical Notch ligand 3 (DLL3), endothelin receptor type B (EDNRB), ephrin A4 (EFNA4), epidermal growth factor receptor (EGFR), EGFRvIll, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), glycoprotein non-metastatic B (GPNMB), guanylate cyclase 2 C (GUCY2C), human epidermal growth factor receptor 2 (HER2), human epidermal growth factor receptor 3 (HERS), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), protein tyrosine phosphatase mu (PTPmu) solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), or trophoblast cell-surface antigen (TROP-2).

Non-limiting examples of antibodies that specifically bind to tumor antigens which may be used as a targeting moiety include Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sotituzumab, Catumaxomab, Ertumaxomab, Depatuxizumab, Ontuxizumab, Blontuvetmab, Tamtuvetmab, or a tumor antigen-binding variant thereof. As used herein, “variant” is meant the antibody specifically binds to the particular antigen (e.g., HER2 for trastuzumab) but has fewer or more amino acids than the parental antibody (e.g., is a fragment (e.g., scFv) of the parental antibody), has one or more amino acid substitutions relative to the parental antibody, or a combination thereof.

By way of example, where the cell targeted comprises an ovarian cancer cell, the targeting moiety can comprise an antibody or peptide to human CA-125R. Over expression of CA-125 has implication in ovarian cancer cells. Alternatively, where the cell targeted comprises a malignant cancer, such as glioblastoma, the targeting moiety can comprise an antibody or peptide to extracellular growth factor receptor (EGFR), human transferrin receptor (TfR), and/or extracellular cleaved PTPmu. Overexpression of EGFR and TfR as well as extracellular cleavage of PTPmu has been implicated in the malignant phenotype of tumor cells.

Other targeting moieties can include a PSMA targeting moiety or PSMA ligand that can selectively recognize PSMA-expressing tumors, cancer cells, and/or cancer neovasculature in vivo. PSMA is a transmembrane protein that is highly overexpressed (100-1000 fold) on almost all prostate cancer (PC) tumors. Only 5-10% of primary PC lesions have been shown to be PSMA-negative. PSMA expression levels increase with higher tumor stage and grade.

Small molecule PSMA ligands bind to the active site in the extracellular domain of PSMA and are internalized and endosomally recycled, leading to enhanced tumor uptake and retention and high image quality. Examples of PSMA ligands are described in Afshar-Oromieh A, Malcher A, Eder M, et al. PET imaging with a [68Ga]gallium-labelled PSMA ligand for the diagnosis of prostate cancer: biodistribution in humans and first evaluation of tumor; Weineisen M, Schottelius M, Simecek J, et al. 68Ga- and 177Lu-Labeled PSMA I&T: Optimization of a PSMA-Targeted Theranostic Concept and First Proof-of-Concept Human Studies. J Nucl Med. 2015; 56:1169-1176. lesions. Eur J Nucl Med Mol Imaging. 2013; 40:486-495; Cho S Y, Gage K L, Mease R C, et al. Biodistribution, tumor detection, and radiation dosimetry of 18F-DCFBC, a low-molecular-weight inhibitor of prostate-specific membrane antigen, in patients with metastatic prostate cancer. J Nucl Med. 2012; 53:1883-1891; and Rowe S P, Gage K L, Faraj S F, et al. (1)(8)F-DCFBC PET/CT for PSMA-Based Detection and Characterization of Primary Prostate Cancer. J Nucl Med.

Other examples of PSMA ligands are described in U.S. Pat. Nos. 6,875,886, 6,933,114, and 8,609,142, which are incorporated herein by reference in their entirety. Still other examples PSMA ligands are disclosed in U.S. Patent Application Publication No. 2015/0366968, U.S. Patent Application Publication No. 2015/0366968, 2018/0064831, 2018/0369385, and U.S. Pat. No. 9,889,199 all of which are incorporated by reference in their entirety.

In some embodiments, the PSMA ligand can have the general formula (I):

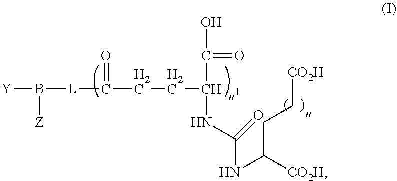

wherein:
n and n¹ are each independently 1, 2, 3, or 4;
L is an optionally substituted aliphatic or heteroaliphatic linking group;
B is linker, such as a peptide linker, that includes at least one negatively charged amino acid; and
Y is a lipid of the nanobubble, which is directly or indirectly linked or coupled to B, and
Z is hydrogen or at least one of a detectable moiety or label or a therapeutic agent, which is directly or indirectly linked or coupled to B. In other embodiments, Z can be selected from the group consisting of an imaging agent, an anticancer agent, or a combination thereof. In still other embodiments, Z is a fluorescent label, such as Rhodamine, IRDye700, IRDye800, Cy3, Cy5, and/or Cy5.5.

Optionally, the cell targeted nanobubbles can include a therapeutic agent that is encapsulated by and/or linked to the membrane. Examples of therapeutic agents can include, but are not limited to, chemotherapeutic agents, biologically active ligands, small molecules, DNA fragments, DNA plasmids, interfering RNA molecules, such as siRNAs, oligonucleotides, and DNA encoding for shRNA. Therapeutic agents can refer to any therapeutic or prophylactic agent used in the treatment (including the prevention, diagnosis, alleviation, or cure) of a malady, affliction, condition, disease or injury in a subject. It will be appreciated that the membrane can additionally or optionally include proteins, carbohydrates, polymers, surfactants, and/or other membrane stabilizing materials, any one or combination of which may be natural, synthetic, or semi-synthetic.

In some embodiments, the therapeutic agent can be at least one of a chemotherapeutic agent, an anti-proliferative agent, an anti-microbial agent, a biocidal agent, and/or a biostatic agent. The therapeutic agent can be encapsulated by and/or linked to the membrane of the nanobubble.

In some embodiments, the cell targeted nanobubbles can be formed by dissolving at least one lipid and a lipid linked to a targeting moiety in propylene glycol. For example, a PSMA targeted nanobubble can be prepared by dissolving 1,2-dibehenoyl-sn-glycero-3-phosphocholine (DBPC, Avanti Polar Lipids Inc., Pelham, Ala.), 1,2-Dipalmitoyl-sn-glycero-3-Phosphate; DPPA, 1,2-dipalmitoyl-sn-glycero-3-phosphor ethanolamine; DPPE (Corden Pharma, Switzerland), and 1,2-distearoyl-sn-glycero-3-phosphoethanolamine-N-[methoxy (polyethylene glycol)-2000] (ammonium salt) (DSPE-mPEG 2000, Laysan Lipids, Arab, Ala.) along with DSPE-PEG-PSMA-1 in propylene glycol to produce a lipid-propylene glycol solution. It will be appreciated that other materials can be dissolved in the propylene glycol, such as proteins, carbohydrates, polymers, surfactants, and/or other membrane stabilizing materials.

After producing the lipid-propylene glycol solution, a glycerol and phosphate buffered solution (PBS) solution can be added to lipid-propylene glycol solution and the resulting solution can be mixed by, for example, sonication. The mixed solution can be transferred to a vial. The air can removed from the sealed vial containing the hydrated lipid solution and replaced with a gas, such as octafluoropropane, until the vial pressure equalized. The resultant solution can then be shaken or stirred for a time (e.g., about 45 seconds) sufficient to form the nanobubbles. In one example, a lipid-propylene glycol solution comprising DBPC/DPPA/DPPE/DSPE-PEG-PSMA-1 dissolved in propylene glycol can be contacted with a hydration PBS/glycerol solution, placed in a vial, and then placed in an incubator-shaker at about 37° C. and at about 120 rpm for about 60 minutes. In some embodiments, the resultant solution containing the nanobubbles can be freeze dried and reconstituted for storage and shipping or frozen and thawed before use.

The cell targeted nanobubbles so formed can be administered to a subject via any known route, such as via an intravenous injection. By way of example, a composition comprising a plurality of octafluoropropane-containing nanobubbles can be intravenously administered to a subject that is known to or suspected of having a tumor.

In some embodiments, the nanobubbles are administered to a subject to treat a neoplastic disease, such as a solid tumor, e.g., a solid carcinoma, sarcoma or lymphoma, and/or an aggregate of neoplastic cells. The tumor may be malignant or benign and can include both cancerous and pre-cancerous cells.

A composition comprising the cell targeted nanobubbles can be formulated for administration (e.g., injection) to a subject diagnosed with at least one neoplastic disorder. For example, the cell targeted nanobubbles can be targeted to prostate cancer cells by conjugating a PSMA ligand that this is specific for the PSMA antigen that is over expressed on prostate cancer cells. The cell targeted nanobubbles can be formulated with at least one lipid that is conjugated to PEG. The nanobubbles can then be combined with the PSMA ligand, which will then become conjugated to PEG of the lipid.

The location(s) where the nanobubble composition is administered to the subject may be determined based on the subject's individual need, such as the location of the neoplastic cells (e.g., the position of a tumor, the size of a tumor, and the location of a tumor on or near a particular organ). For example, the composition may be injected intravenously into the subject. It will be appreciated that other routes of injection may be used including, for example, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal routes.

The cell targeted nanobubbles administered to the subject can circulate in the subject and bind to and/or complex with the targeted cells by binding and/or complexing of the targeting moiety with the cell surface molecule of the targeted cell. Typically, the cell targeted nanobubbles can bind to and/or complex with the targeted cells within about 10 minutes, about 15 minutes, about 20 minutes, about 25 minutes, about 30 minutes, about 35 minutes, about 40 minutes, about 45 minutes, about 50 minutes, about 55 minutes, or about 1 hour or less.

Once the cell targeted nanobubbles are bound to and/or complexed with the target cell, the size and/or diameter of the cell targeted nanobubbles allows the nanobubbles to be internalized by or enter the targeted cell by, for example, endocytosis and/or phagocytosis. The cell targeted nanobubbles can accumulate within the targeted cell and remain within the cells for an extended period, for example, at least one hour, two hours, three hours, or more.

Referring again to FIG. 1 and FIG. 2, following internalization of cell targeted nanobubbles into the target cell, at step 14 of the method 10, the internalized nanobubbles can be insonated with ultrasound energy of a given frequency, acoustic pressure, and time effective to promote violent oscillation, vibration, and rapid volumetric collapse and/or inertial cavitation of the internalized nanobubbles resulting in apoptosis and/or necrosis of the target cell.

Insonation of the internalized nanobubbles can be achieved by using a non-invasive, minimally invasive, and/or external ultrasound source that produces ultrasound energy effective to promote inertial cavitation. The intensity and frequency of the applied ultrasound signal, as well as the duty cycle and pattern for activating the ultrasound source are controllable and configured to suit a given application. Monitoring nanobubble dynamics and correlating signatures of inertial collapse with treatment parameters presents a strategy for gaining further insights on the mechanism of action as well as intra-treatment monitoring for improving clinical outcomes.

The ultrasound source can provide specific acoustic sequences that can drive nanobubble collapse without disrupting and/or adversely affecting normal cells and tissues. These sequences can be applied from a non-focused transducer, which is distinct from typical focused ultrasound transducers used for drug delivery and ultrasound therapy, such as histotripsy. The use of non-focused ultrasound makes it possible to treat lesions like wide-spread cancer micrometastasis, for example, in liver or bone, which cannot be easily visualized and thus on which focused ultrasound cannot be used. It will be appreciated that focused transducers can also be used for specific applications.

In some embodiments, the insonation can be at a duty cycle of about 1% to about 50%, about 1% to about 40%, about 1% to about 30%, about 1% to about 25%, about 1% to about 20%, about 1% to about 15%, or about 5% to about 15%, an ultrasound frequency of about 1 MHz to about 50 MHz, 1 MHz to about 40 MHz, 1 MHz to about 30 MHz, 1 MHz to about 20 MHz, 1 MHz to about 15 MHz, or about 1 MHz to about 10 MHz, an intensity of about 0.1 W/cm² to about 10 W/cm², about 0.1 W/cm² to about 9 W/cm², about 0.1 W/cm² to about 8 W/cm², about 0.1 W/cm² to about 7 W/cm², about 0.1 W/cm² to about 6 W/cm², about 0.1 W/cm² to about 5 W/cm², about 0.1 W/cm² to about 4 W/cm², or about 1 W/cm² to about 4 W/cm², a pressure amplitude of about 50 kPa to about 1 MPa, about 50 kPa to about 900 KPa, about 50 kPa to about 800 KPa, about 50 kPa to about 750 KPa, about 100 kPa to about 750 KPa, or about 150 kPa to about 750 KPa, and a time of about 1 minute to about 30 minutes, about 1 minute to about 25 minutes, about 1 minute to about 20 minutes, about 1 minute to about 15 minutes, about 1 minute to about 10 minutes, or about 1 minute to about 5 minutes.

In other embodiments, the insonation can include two ultrasound pulse sequences with pulses of different pressure amplitudes sent to tissue in which the nanobubbles are administered, wherein one pulse has a pressure amplitude greater than the other pulse. For example, one pulse can have a pressure amplitude at least twice the other pulse.

In some embodiments, one pulse is below the nanobubble pressure threshold for inertial cavitation followed by one above the threshold pressure threshold for inertial cavitation. For example, for a nanobubble with a pressure threshold of 200 kPA, the first pulse is 150 kPA, and is followed by one at 250 kPa. In another example, for a nanobubble with a pressure threshold of 500 kPa, one pulse is 300 kPA and second is 600 kPa.

In other embodiments, to induce maximum inertial cavitation, the overall pulse length may also be longer (10-30 cycles) than a typical imaging pulse (3-6 cycles).

In some embodiments, a system that includes the ultrasound source may also be equipped with both the ultrasound source (transmitter) as well as a passive cavitation detection and monitoring acoustic sensor (receiver). The acoustic sensor may be integrated into the transmitting ultrasound source as a transducer element in an array of a plurality of elements, or the acoustic sensor may be implemented as a stand-alone sensor, such as a hydrophone which is suitably placed with respect to the ultrasound source and target region. Instead of simply detecting reflected ultrasound signals at the source frequency, this system can rely on detection of inertial cavitation signals arising from the collapse of cell targeted nanobubbles that selectively accumulate the targeted cells.

In some embodiments, the therapy and/or methods described herein can be used to treat cancer in a subject in need thereof. Such methods include administering to the subject a plurality of cancer cell targeted nanobubbles. Each of the cancer cell targeted nanobubbles can have a lipid membrane that defines at least one internal void, which includes at least one gas, and a targeting moiety that is linked to an external surface of the lipid membrane. The targeting moiety can bind to a cancer cell surface molecule of a target cancer cell. The cancer cell targeted nanobubbles can have a size and/or diameter that facilitates internalization of the nanobubbles by the target cancer cell upon binding of the targeting moiety to the cancer cell surface molecule. Following administration of the cancer cell targeted nanobubbles to the subject and internalization of the cancer cell targeted nanobubbles into the cancer cells, the internalized nanobubbles can be insonated with ultrasound energy effective to promote inertial cavitation of the internalized nanobubbles and apoptosis and/or necrosis of the target cancer cell.

In certain embodiments, the subject has a cancer characterized by the presence of a solid tumor, a semi-solid tumor, a primary tumor, a metastatic tumor, a liquid tumor (e.g., a leukemia or lymphoma), and/or the like. Cancers, which can be treated using the methods described herein, include, but are not limited to, adult and pediatric acute lymphoblastic leukemia, acute myeloid leukemia, adrenocortical carcinoma, AIDS-related cancers, anal cancer, cancer of the appendix, astrocytoma, basal cell carcinoma, bile duct cancer, bladder cancer, bone cancer, biliary tract cancer, osteosarcoma, fibrous histiocytoma, brain cancer, brain stem glioma, cerebellar astrocytoma, malignant glioma, glioblastoma, ependymoma, medulloblastoma, supratentorial primitive neuroectodermal tumors, hypothalamic glioma, breast cancer, male breast cancer, bronchial adenomas, Burkitt lymphoma, carcinoid tumor, carcinoma of unknown origin, central nervous system lymphoma, cerebellar astrocytoma, malignant glioma, cervical cancer, childhood cancers, chronic lymphocytic leukemia, chronic myelogenous leukemia, acute lymphocytic and myelogenous leukemia, chronic myeloproliferative disorders, colorectal cancer, cutaneous T-cell lymphoma, endometrial cancer, ependymoma, esophageal cancer, Ewing family tumors, extracranial germ cell tumor, extragonadal germ cell tumor, extrahepatic bile duct cancer, intraocular melanoma, retinoblastoma, gallbladder cancer, gastric cancer, gastrointestinal stromal tumor, extracranial germ cell tumor, extragonadal germ cell tumor, ovarian germ cell tumor, gestational trophoblastic tumor, glioma, hairy cell leukemia, head and neck cancer, hepatocellular cancer, Hodgkin lymphoma, non-Hodgkin lymphoma, hypopharyngeal cancer, hypothalamic and visual pathway glioma, intraocular melanoma, islet cell tumors, Kaposi sarcoma, kidney cancer, renal cell cancer, laryngeal cancer, lip and oral cavity cancer, small cell lung cancer, non-small cell lung cancer, primary central nervous system lymphoma, Waldenstrom macroglobulinema, malignant fibrous histiocytoma, medulloblastoma, melanoma, Merkel cell carcinoma, malignant mesothelioma, squamous neck cancer, multiple endocrine neoplasia syndrome, multiple myeloma, mycosis fungoides, myelodysplastic syndromes, myeloproliferative disorders, chronic myeloproliferative disorders, nasal cavity and paranasal sinus cancer, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary cancer, plasma cell neoplasms, pleuropulmonary blastoma, prostate cancer, rectal cancer, rhabdomyosarcoma, salivary gland cancer, soft tissue sarcoma, uterine sarcoma, Sezary syndrome, non-melanoma skin cancer, small intestine cancer, squamous cell carcinoma, squamous neck cancer, supratentorial primitive neuroectodermal tumors, testicular cancer, throat cancer, thymoma and thymic carcinoma, thyroid cancer, transitional cell cancer, trophoblastic tumors, urethral cancer, uterine cancer, uterine sarcoma, vaginal cancer, vulvar cancer, choriocarcinoma, hematological neoplasm, adult T-cell leukemia, lymphoma, lymphocytic lymphoma, stromal tumors and germ cell tumors, or Wilms tumor. In some embodiments, the cancer is lung cancer, breast cancer, prostate cancer, colorectal cancer, gastric cancer, liver cancer, pancreatic cancer, brain and central nervous system cancer, skin cancer, ovarian cancer, leukemia, endometrial cancer, bone, cartilage and soft tissue sarcoma, lymphoma, neuroblastoma, nephroblastoma, retinoblastoma, or gonadal germ cell tumor.

In some embodiments, the subject has a cancer selected from breast cancer, glioblastoma, neuroblastoma, head and neck cancer, gastric cancer, ovarian cancer, skin cancer (e.g., basal cell carcinoma, melanoma, or the like), lung cancer, colorectal cancer, prostate cancer, glioma, bladder cancer, endometrial cancer, kidney cancer, leukemia (e.g., T-cell acute lymphoblastic leukemia (T-ALL), acute myeloid leukemia (AML), etc.), liver cancer (e.g., hepatocellular carcinoma (HCC), such as primary or recurrent HCC), a B-cell malignancy (e.g., non-Hodgkin lymphomas (NHL), chronic lymphocytic leukemia (CLL), follicular lymphoma, mantle cell lymphoma, diffuse large B-cell lymphoma, and the like), pancreatic cancer, thyroid cancer, any combinations thereof, and any sub-types thereof.

A pharmaceutical composition comprising the cancer cell targeted nanobubbles described herein can be administered to the subject in a therapeutically effective amount. In some embodiments, a therapeutically effective amount of the cancer cell targeted nanobubbles is an amount that, when administered alone (e.g., in monotherapy) or in combination (e.g., in combination therapy) with one or more additional therapeutic agents, in one or more doses, is effective to reduce the symptoms of the pathological condition (e.g., cancer) in the individual by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptoms in the individual in the absence of treatment with the conjugate. According to some embodiments, when the subject has cancer, the methods described herein promote apoptosis and/or necrosis of the cancer when the cancer cell targeted nanobubbles are administered in an effective amount.

Dosing is dependent on severity and responsiveness of the condition (e.g., cancer) to be treated. Optimal dosing schedules can be calculated from measurements of drug accumulation in the body of the individual. The administering physician can determine optimum dosages, dosing methodologies and repetition rates. Optimum dosages may vary depending on the relative potency of individual agent and can generally be estimated based on EC50s found to be effective in in vitro and in vivo animal models, etc. In general, dosage may be given once or more daily, weekly, monthly, or yearly. The treating physician can estimate repetition rates for dosing based on measured residence times and concentrations of the conjugate in bodily fluids or tissues. Following successful treatment, it may be desirable to have the subject undergo maintenance therapy to prevent the recurrence of the disease state, where the cell targeted nanobubbles can be administered in maintenance doses once or more daily, to once every several months, once every six months, once every year, or at any other suitable frequency.

In some embodiments, a drug, and/or therapeutic agent, such as a chemotherapeutic (e.g., doxorubicin) can also be loaded into the nanobubble during nanobubble formation to provide a drug loaded cell targeted nanobubble. The drug loaded cell targeted nanobubble can be responsive to the ultrasound energy to release the therapeutic agent from the nanobubble administering the nanobubble to a subject. Advantageously, cell targeted nanobubbles that allow remote release of the therapeutic agent, such as a chemotherapeutic agent (e.g., doxorubicin) can target or be targeted to specific cells or tissue of subject, such as tumors, cancers, and metastases, by systemic administration (e.g., intravenous, intravascular, or intraarterial infusion) to the subject and once targeted to the cells or tissue remotely released to specifically treat the targeted cells or tissue of subject (e.g., tumors, cancers, and metastasis). Targeting, inertial cavitation of the nanobubbles, and selective release of the chemotherapeutic agents to malignant cancer metastases allows treatment of such metastases using chemotherapeutics, which would provide an otherwise negligible effect if not targeted and remotely released using the nanobubbles described herein.

The cell targeted nanobubbles can allow the combination of any of the above noted therapeutic agents and therapies to be administered at a low dose, that is, at a dose lower than has been conventionally used in clinical situations.

A benefit of lowering the dose of the combination therapeutic agents and therapies administered to a subject includes a decrease in the incidence of adverse effects associated with higher dosages. For example, by the lowering the dosage of a chemotherapeutic agent, such as doxorubicin, a reduction in the frequency and the severity of nausea and vomiting will result when compared to that observed at higher dosages. Similar benefits are contemplated for the compounds, compositions, agents and therapies in combination with the nanobubbles.

By lowering the incidence of adverse effects, an improvement in the quality of life of a patient undergoing treatment for cancer is contemplated. Further benefits of lowering the incidence of adverse effects include an improvement in patient compliance, a reduction in the number of hospitalizations needed for the treatment of adverse effects, and a reduction in the administration of analgesic agents needed to treat pain associated with the adverse effects.

It will be appreciated that the cell targeted nanobubbles and methods described herein can be used in other applications besides diagnostic, therapeutic, and theranostic applications described above. For example, the cell targeted nanobubble can be targeted to cells of microorganisms, such as bacteria and fungus, and insonated upon internalization of the cells to promote inertial cavitation of the nanobubbles and treat infections in a subject and particularly infections that are resistant to antimicrobial agents. Advantageously, the cell targeted nanobubbles can also deliver biocidal agents and/or biostatic agents that that kill microbes as well as agents that simply inhibit their growth or accumulation.

The following example is for the purpose of illustration only and is not intended to limit the scope of the claims, which are appended hereto.

### Example 1

In this Example we investigated the kinetics of PSMA-targeted NB distribution with high frequency ultrasound across the entire tumor volume and examined the differences in contrast agent dynamic in the tumor rim and tumor core. This example also further shows that PSMA-NB extravasation and accumulation in whole tumor mass using three dimensional (3D) US imaging.

We previously showed that active targeting to PSMA enhances tumor uptake of intact PSMA-NBs with extended retention, which results in prolonged US signal enhancement in the tumors over 25 min that can be visualized with clinical nonlinear ultrasound. One hypothesis for the prolonged tumor enhancement is that PSMA-targeted NBs are internalized into their target cancer cells and the internalization delays octafluoropropane gas dissolution.

This example also shows the effect of receptor-mediated endocytosis of PSMA-NBs on their acoustic activity and intracellular persistence using an in vitro cellular model. Elucidating the mechanism of interaction of PSMA-NB at the cellular level can offer a new avenue to clinical translation of PSMA-NB in PCa diagnosis and therapeutic applications.

### Experimental Section/Methods

### NB Formulation and Characterization

Lipid shell-stabilized C₃F₈ NB functionalized with the PSMA-1 ligand were formulated as previously reported. Briefly, a cocktail of lipids including DBPC, DPPE, DPPA, mPEG-DSPE, and DSPE-PEG-PSMA-1 were dissolved in propylene glycol, glycerol and PBS. This was followed by gas exchange with C₃F₈, mechanical agitation, and centrifugation after which the NBs were isolated. PSMA-NB and NB were characterized as previously described.

### Animal Model

Animals were handled according to a protocol approved by the Institutional Animal Care and Use Committee (IACUC) at Case Western Reserve University and were in accordance with all applicable protocols and guidelines in regards to animal use. Male athymic nude mice (4-6 weeks old) were anesthetized with inhalation of 3% isoflurane with 1 L/min oxygen and were implanted subcutaneously with 1×10⁶ of PSMA-negative-PC3flu and PSMA-positive-PC3pip cells in 100 μL matrigel. Animals were observed every other day until tumors reached at about 8-10 mm in diameter.

### In Vivo NLC Imaging for Bubble Kinetic Analysis

In vivo experiments were performed using FUJIFILM VisualSonic Vevo 3100. Total 9 animals were used for the experiment. Animals were divided into 3 groups; PSMA-NB, NB and Lumason MB group. In vivo bubble distribution was imaged with 2D non-linear contrast mode. A total volume of 200 μl of undiluted either PSMA-NB or NB were injected via tail vein. To obtain wash-in bubble dynamic tumor scanned for ˜3 min at 5 fps. The scanning parameters were setup to 18 MHz frequency, with MS250 transducer, 4% transmit power, 30 dB contrast gain, medium beam width, 40 dB dynamic range. Then 3D US scan was then performed at the peak signal followed by non-linear contrast imaging was accomplished to see the kinetic of washout phase at lfps and 1000 frames for ˜16 min with maintaining above parameters during the imaging session.

Whole Tumor Imaging with 3D Ultrasound

Ultrasound 3D tumor imaging was performed with Vevo 3100 (FUJIFILM, Visual Sonics) scanner. Transducer was clipped onto the 3D motor and positioned onto the tumor area. By adjusting the X-Y axis position of the probe, placed the probe at the center of the tumor as the imaging display. The 3D setup arranged to make 0.05 mm size thickness 2D slices with 383 frames Images obtained and constructed together to obtain the 3D volume to achieve whole tumor bubble distribution Animals were divided into 3 groups; PSMA-NB, NB and Lumason MB group. A total volume of 200 μl of undiluted either PSMA-NB or NB were injected via tail vein. To obtain wash-in bubble dynamic tumor scanned for ˜3 min at 5 fps with same parameters as above. Then 3D scanning was applied to visualize the bubble distribution in whole tumor at the peak contrast signal. After bubble was allowed to freely circulate without US scan and 3D scan was performed again 25 min post injection. To confirm intact bubbles extravagated and accumulated in tumor 3D burst sequence was applied to entire tumor and rescanned to obtain 3D image.

Tumor Extravasation Studies with 3D Ultrasound

For the extravasation studies, animals were divided into 3 groups: PSMA-NB, NNB and Lumason, and 3 animals was used for each group (total n=9). 200 μl of contrast agent was injected via tail vein. Mouse was subjected to 3D US scan 25 min post injection as described previously. Then cardiac perfusion was performed with 50 ml of PBS through the left ventricle and 3D US scan was completed again to detect the US signal produced from intact bubbles that accumulated in the perfused tumor.

### Histology Analysis

Animals were divided into 3-groups: Cy5.5-PSMA-NB (n=3), Cy5.5-NB (n=3), and no-contrast-control. Cy5.5 labeled NBs were prepared by mixing DSPE-PEG-Cy5.5 (100 μl) into the lipid solution. Mice received either 200 □l of undiluted UCAs or PBS via tail-vein. 25 min after injection, animals were scanned using US to detect the signal and then PBS perfusion was performed with 50 ml-PBS though left-ventricle. Then, tumors were scan again to perceive the US signal that generate from intact-NB. Tumors and the kidney were harvested, fixed in paraformaldehyde and embedded in optimal-cutting-temperature compound (OCT Sakura Finetek USA Inc., Torrance, Calif.). The tissues were cut into 8 μm slices and washed (3×) with PBS and incubate with protein blocking solution that contain 0.5% TritonX-100 (Fisher Scientific, Hampton, N.H.) and incubated in 1:250 diluted primary-antibody CD31(PECAM-1) Monoclonal Antibody Fisher Scientific, Hampton, N.H.) for 24h at 4° C. It was then washed with PBS, incubated with Alexa-568 tagged secondary-antibody (Fisher Scientific, Hampton, N.H.) for 1 h, and stained with DAPI (Vector Laboratories, Burlingame, Calif.). The fluorescence images were obtained and analyzed (by interactive function of segmentation and threshold) using Axio Vision V 4.8.1.0, Carl Zeiss software (Thornwood, N.Y.). For PSMA-immunohistochemistry, tissues were wash 3× with PBS and incubated with blocking solution followed by 1:150 diluted PSMA primary-antibody (Thermo Fisher Scientific, Waltham, Mass.) for 24h at 4° C. and followed the above steps as for CD31 staining.

### Preparation and Characterization of Contrast Agents

The preparation and characterization of NBs has been reported elsewhere. Briefly, a cocktail of lipids including DBPC (Avanti Polar Lipids Inc., Pelham, Ala.), DPPE, DPPA (Corden Pharma, Switzerland), and mPEG-DSPE2000 (Laysan Lipids, Arab, Ala.) were dissolved in propylene glycol (PG, Sigma Aldrich, Milwaukee, Wis.), glycerol and PBS. Then gas exchanged with C3F8 (Electronic Fluorocarbons, LLC, PA) and vial was subjected to mechanical agitation. NBs were isolated by centrifugation. PSMA targeted NB formulated by incorporating DSPE-PEG-PSMA-1 into the lipid cocktail mixture. PSMA-NB and NB were characterized as previously described.

### Cell Culture Studies

Retrovirally transformed PSMA-positive PC3pip cells and PC3flu cells (transfection-control) were originally obtained from Dr. Michel Sadelain (Memorial-Sloan Kettering Cancer Center, New York, N.Y.). Cell lines were checked and authenticated by western blot. Cells were grown in complete RPMI1640 medium (Invitrogen Life Technology, Grand Island, N.Y.) at 37° C. and 5% CO₂ environment.

### Cellular Uptake Studies

Both PC3pip and PC3flu cells (2×10⁶ cells/ml) were plated on cell culture petri dishes (60×15 mm, Fisher Scientific) at about 70% confluence. Twenty-four hours later, cells were incubated with PSMA-NB or plain NBs (˜10,000 bubbles/cell) for 1 h. After incubation, cells were washed three times with PBS (3×) and maintained in RPMI at 37° C. until the US scan time points. Before US scan cells were trypsinized, counted and 1×10⁶ cells were used.

### Acoustic Assessment of PSMA Targeted NB Internalized in PC3pip Cells In Vitro

In vitro acoustic activity of NBs internalized cells was assessed using a clinical US scanner (AplioXG SSA-790A, Toshiba Medical, now Hitachi Healthcare America). To carry out the measurements, cells (˜2×10⁶) were washed and detached using trypsin. Following detachment and resuspension in PBS, the cell suspension was placed in a custom-made 1.5% (w/v) agarose phantom.^{[31]} The phantom was affixed over a 12 MHz linear array transducer, and images were acquired with contrast harmonic imaging (CHI) at 0.1 mechanical index (MI), 65 dynamic range, 70 dB gain, and 0.2 frames/sec imaging frame rate. Using onboard software, a region of interest (ROI) analysis was performed on all samples to measure the mean signal intensity in each ROI. The data were then exported to Microsoft Excel for further processing. The experiments were carried out in triplicate.

### Confocal Imaging of PSMA-NB Internalized PC3pip Cells

PC3pip cells were seeded in glass bottom petri dishes (MetTek Corporation, Ashland, Mass., USA) at a density of 10⁴ cells/well. Rhodamine-labeled-NBs were prepared by mixing DSPE-Rhodamine (50 □1) into the lipid solution. After 24 h, 1:10 diluted Rhodamine-tagged PSMA-NB (250 μL) were added to the cells for 1 h. Following incubation, cells were washed with PBS and were then placed into the incubator in RPMI for 3 h and for 24 h. The 3 h time-point was chosen because a significantly high acoustic activity was previously observed with PSMA-NB internalized PC3pip cells with low standard error at the 3 h time point. One hour before the end of incubation 5 μM Lysotracker Red 24 μL), a marker for late endosomes and lysosomes, (ThermoFisher Scientific) was added the cells, as per manufacturer instructions. After incubation, cells were washed 3× with PBS and fixed with 4% paraformaldehyde for 10 min Cells were washed with PBS and stained with DAPI mounting medium (Vecor Laboratories, Burlingame, Calif.). Then cells were observed using a fluorescent microscope (Leica DMI 4000B, Wetzlar, Germany) equipped with the appropriate filter sets. LysoTracker Red exhibits green fluorescence (excitation: 577 nm, emission: 590 nm).

### Analysis of Octafluoropropane Gas Trapped in Cells Using GC/MS

Presence of octafluoropropane (C₃F₈) gas inside cells was confirmed by headspace gas chromatography/mass spectrometry (GC/MS) as previously described. For these experiments, cells (1×10⁷ cell/mL) were grown in cell culture flasks (75 cm² size) for 24 h. Again, as above, cells were then incubated with 1 mL of either PSMA-NB or plain NB (˜10000 NBs/cell) for 1 h. After incubation, cells were washed with PBS and incubated in medium for 3 h. Following incubation, the cells were trypsinized, re-suspended in PBS and centrifuged at 1000 rpm for 4 min. Then the cells were then transferred to GC headspace vials with 300 μL of medium and 300 μL of cell lysis buffer, sealed with PTFE/silicon septum and capped (Thermos Fisher Scientific). The vials were sonicated for 20 min in an ultrasonic water bath (Branson Ultrasonics, Danbury, Conn.) at 50° C. to release C₃F₈ gas into the headspace vial. The GC/MS analysis was performed as described previously using the Agilent 5977B-MSD equipped mass spectrometer with an Agilent 7890B gas chromatograph GC/MS system. A DB5-MS capillary column (30 m×0.25 mm×0.25 μm) was used with a helium flow of 1.5 mL/min. Headspace samples of 1 μL were injected at 1:10 split. Gas chromatography conditions used were as follows: oven temperature was at 60° C., held for 1 min, ramp 40° C./min until 120° C. and held for 3.5 min. Perfluoropropane eluted at 1.2 min. Samples were analyzed in Selected Ion Monitoring (SIM) mode using electron impact ionization (EI). M/z of 169 (M-19) was used in all analyses. Ion dwell time was set to 10 ms. Perfluoropropane was verified by NIST MS spectra database. The standard calibration plot was obtained by measuring the peak area of GC peak as a function of NB concentrations (0-100×10⁸ NB/mL).

### In Vivo US Imaging of Internalized Bubbles

Animals were handled according to the protocol approved by the Institutional Animal Care and Use Committee (IACUC) at Case Western Reserve University and were in accordance with all applicable protocols and guidelines in regards to animal use. PC3pip cells were incubated with PSMA-NB and processed as described above. Mice (n=9, 4-6 weeks old Athymic (NCR nu/nu) mice with 20 g weight) were randomly divided into 3 groups and were anesthetized with inhalation of 3% isoflurane with 1 L/min oxygen. Baseline US signal was obtained both left and right side of flank area (marked with a permanent marker) using the parameters described above at 0.1 and 0.5 mechanical index (MI). Following incubation with PSMA-NB, the PC3pip cells were suspended in a Matrigel and PBS mixture (1:1 PBS/Matrigel) and cell suspension (100 μl) was injected subcutaneously into flank of nude mice. As a control, cells without NB exposure were injected adjacent to the NB-exposed cells (FIG. 7A). US images of the injection sites were obtained immediately after injection at MI=0.1 immediately or after 3, 24 h or 8 days using the same parameters as above. After imaging at 0.1 MI, the MI value was increased to 0.5 and regions were imaged again for all time points. The region of interest (ROI) was drawn around the injected cell-region, excluding the skin. Then mean signal intensity for each ROI was obtained from the CHIQ software. These measurements were exported to Excel and the signal enhancement in each ROI by subtracting the baseline value (contrast before the inoculation).

### Results

### Nanobubble Characterization

Nanobubble preparation, functionalization with the PSMA-1 ligand and verification of the lipid-ligand conjugation have been reported previously. The diameter of NB and PSMA-NB as characterized by resonant mass measurement (RMM) was 281□2 nm and 277 □11 nm, respectively. Validation of the RMM analysis and its optimization for use in NB characterization has been previously described. Importantly, results show that the mean size and concentration did not change significantly after functionalization (the concentration of NB and PSMA-NB was 4E11 □2.45E10 and 3.9E11 □2.82E10 NB/ml, respectively).

### Ultrasound Signal in Tumor Rim and Core

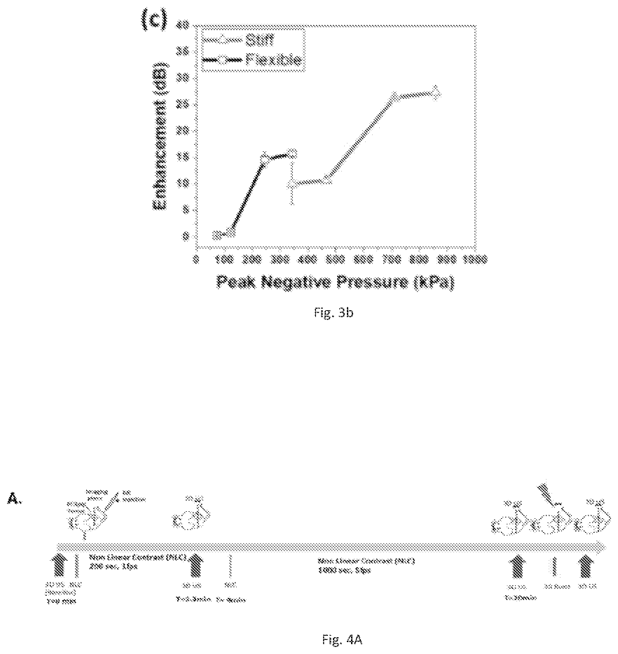

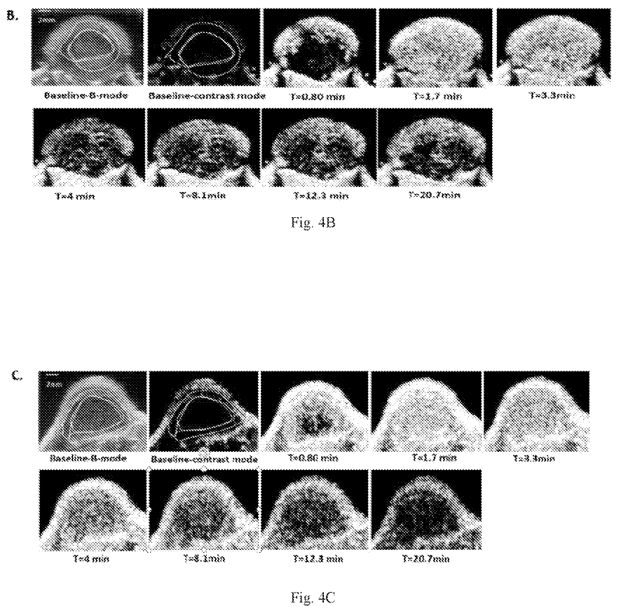

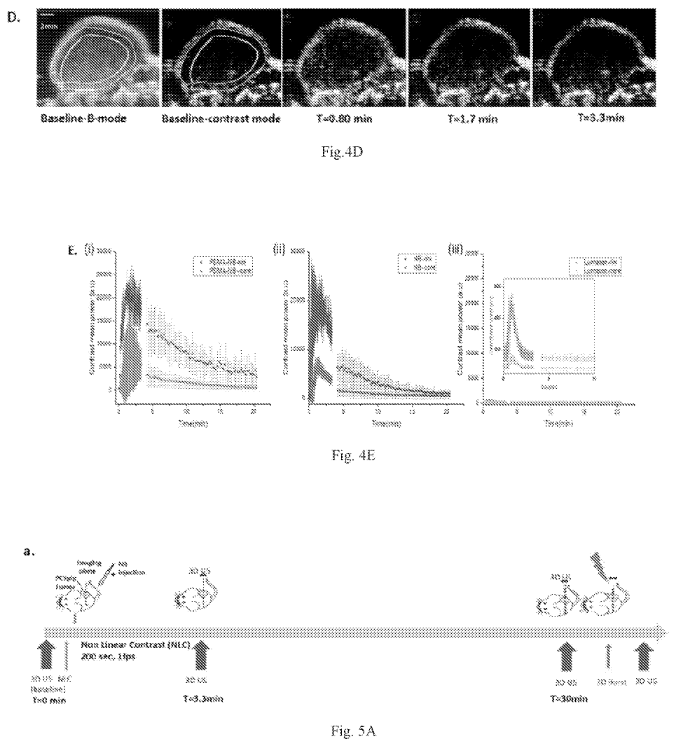

In vivo experiments were performed using FUJIFILM VisualSonic Vevo 3100. A total of 9 tumor bearing mice were used for the experiment. Animals were divided into 3 groups: PSMA-NB, NB and Lumason MB group. In vivo bubble distribution was imaged with 2D non-linear contrast mode after injecting a total volume of 200 μl of undiluted either PSMA-NB or NB via tail vein. FIG. 4A shows the schematic diagram of time-line of the ultrasound scan process. A baseline scan was performed with both non-linear contrast mode and the 3D mode before bubble injection. To obtain wash-in bubble dynamic tumor was scanned for ˜3 min at 5 frames per second (fps). FIG. 4B, C, D. FIG. 4E demonstrates the TIC curves corresponding to the tumor rim and core obtained with the first 1000 frames at 5 fps followed by a second scan of 1000 frames at 1 fps. Before changed the frame rates from 5 fps to 1 fps, a 3D US scanned was performed to observe the bubble distribution in the whole tumor mass.

Rapid signal enhancement was observed in tumors imaged with either PSMA-NB or plain NB approximately, reaching peak intensity in 1 to 2 min post-injection. There was no significant difference in peak enhancement (PE) of entire tumor for PSMA-NB and NB, which was an indication of the similar morphology of targeted and untargeted bubbles. The variability of bubble kinetic parameters was further investigated by analyzing the tumor rim and core separately. A loop ROI was drawn to the tumor rim separately to distinguish from the tumor core (FIG. 4). As shown in FIG. 4E, immediately after injection, PSMA-NB and plain NB filled up the tumor rim rapidly. In contrast, PSMA-NB demonstrated slower wash-in to the tumor core compared to the tumor rim. The kinetic parameters were calculated and summarized in Table 1. The time to peak (TTP) to tumor core for the PSMA-NB was significantly greater than that of plain NB (2.48 □0.71 min vs 1.21 □0.15 min, p<0.05). However, the time to peak (TTP) for tumor rim for PSMA-NB and NB was not significantly different. There was no significant difference in wash-in area under the curve (WiAUC) for both groups. Furthermore, the peak enhancement (PE) for tumor rim with both NB and PSMA-NB was not significantly different. Also, the PE for tumor core with both NB and PSMA-NB also not significantly different. The comparable WiAUC and PE indicated the similar kinetic of both bubbles. However, the PE for tumor rim and core was significantly different for both NB group (Table 1).

TABLE 1
Summary of kinetic parameters of
tumor rim and core obtained from time intensity curve (TIC)

| | | Peak | Time to | Wash-in | | |
|---|---|---|---|---|---|---|
| | | Enhancement | peak | AUC | Washout | Total |
| Bubble Type | | (PE) (a.u) | (TTP)(min) | (WiAUC) | AUC | AUC |
| PSMA-NB | Rim | 26263 ± 1930* | 1.77 ± 0.71 | 28718 ± 12677 | 160452 ± 45851* | 187171 ± 48812*^{$} |
| PSMA-NB | Core | 9175 ± 7871* | 2.48 ± 0.71* | 9253 ± 5744 | 37338 ± 23927* | 46591 ± 29509* |
| NB | Rim | 25099 ± 7109^{#} | 1.52 ± 0.62 | 29354 ± 14462* | 76357 ± 23970^{#$} | 103940 ± 17633^{#$} |
| NB | Core | 7445 ± 1963^{#} | 1.21 ± 0.15* | 5142 ± 2306* | 24461 ± 11983^{#} | 29603 ± 9847^{#} |
| Lumason | Rim | 559 ± 118^{α} | 0.84 ± 0.07 | 170 ± 19^{α} | 3013 ± 421^{α} | 3175 ± 426^{α} |
| Lumason | Core | 157 ± 28^{α} | 0.77 ± 0.11 | 59 ± 18^{α} | 1556 ± 170^{α} | 1610 ± 168^{α} |

All values express as mean □ s.d., *, #, $, □
statistically significant different in each group; p < 0.05.

Moreover, the NB accumulation was compared with the commercially available MB contrast agent Lumason. The contrast enhancement occurred rapidly with Lumason (TTP is 0.84 □0.06 min for rim and 0.77 □0.11 for core) and it was significantly different from that of both kinds of NBs. Furthermore, the PE and the WiAUC of Lumason were significantly lower compared to the two NB groups. After peak enhancement, the US signal decayed with the time in both PSMA-NB and NB groups (FIG. 4E). The washout AUC for tumor rim with PSMA-NB was significantly high compared to that of the NB group (Table 1). Similarly, washout AUC for tumor core with PSMA-NB and NB also significantly different. Furthermore, the washout AUC for tumor rim and core was significantly different for both groups. Importantly, the nonlinear contrast imaging parameters used for bubble studies were kept constant, meaning that the transmit frequency used to image Lumason was higher than typically utilized for this agent. This could result in lower sensitivity of detection and subsequent reduction in signal enhancement. However, relative contrast agent kinetics should be relatively unaffected.

The large observed differences in NB dynamics between the rim and core of each tumor may be due to the vascularity inconsistency in the tumor rim and the core due to angiogenesis. Angiogenesis, one of the critical steps during tumor development and progression, contributes to the formation of new capillaries from preexisting blood vessels, as well as promotes tumor growth and metastasis by providing essential nutrients and oxygen to tumor. Also, the PSMA biomarker distribution in the tumor mass also plays an important role on targeted bubble accumulation in tumor. Furthermore, it has reported that PSMA expression is present in the neovasculature endothelial cells. One assumption for the difference in TTP is that the presence of the biomarker that controls the contrast agent flow. The PSMA targeted NB tend to bind the biomarker in the tumor and slow down the flow rate. Hence, the time taken to fill the tumor with PSMA-NB is slower compared to the freely flowing NB. After reaching the peak signal, the contrast of both types of NBs starts to decrease in both tumor rim and the core. However, the washout AUC for PSMA-NB was significantly higher than that of NB indicating high retention of targeted NB in the tumor. The NB signal decrease was most probably due to the disappearance of NB from the tumor rim and the tumor matrix due to tumor interstitial pressure (TIP). In addition to the abnormal vascular formation, the poor lymphatic drainage is elevated in tumor tissues compared to normal tissues, which makes TIP in hindering drug and nanoparticle delivery. Due to the binding of PSMA targeted NB into the PSMA biomarker, the removal by the TIP might be minimized for PSMA-NB.

Whole Tumor Imaging with 3D Ultrasound

To gain a better understanding of the bubble distribution in the whole tumor mass, 3D nonlinear contrast US was implemented after administration of PSMA-NB, NB, and Lumason MB as an extension of 2D imaging. The 0.05 mm size 2D US image slices of the tumor were constructed together to obtain the whole tumor bubble distribution. After nonlinear contrast scanning, 3D US was performed to obtain the contrast signal at the peak in whole tumor mass (FIG. 5). The 3D analysis calculates the percent of voxels which display nonlinear signal within the imaging volume. In agreement with 2D scanning, the 3D analysis also demonstrated a similar signal at the peak for both PSMA-NB and NB (FIGS. 5B and C). At 3 min, both PSMA-NB and NB covered about ˜90% of the tumor rim (86.9±0.8% and 87.7±6.6% respectively, p=0.6). Similarly, PSMA-NB and NB were detected in ˜60% of the tumor core (64.9±14.5% and 62.4±28.1% respectively). The percent of agent of Lumason detected in tumor rim was significantly lower compared to the PSMA-NB and NB. At 3 min, Lumason was detected in 10% (5.7±2.1%) of the tumor rim.

Interestingly, when tumors were imaged with continuous nonlinear ultrasound (1 fps for 16 min after the 3D acquisition) was applied, there was no significant difference agent coverage observed between PSMA-NB and NB groups. Also, the ratio of PSMA-NB and NB signal in the whole tumor was lower compared to the previous observation with 2D scanning We speculate that the continuous exposure of NBs which were immobilized within the tumor tissue to the US with may have resulted in rapid bubble dissolution. To test this hypothesis, another set of experiments was carried out, where NBs, PSMA-NBs or Lumason were injected into the mouse, but then were permitted to circulate for 30 min without US exposure. In these experiments, PSMA-NB showed significantly higher percentage of nonlinear signal in the tumor core compared to both NB and Lumason (25.2±1.5%, 13.9±5.1%, and 0.4±0.4% respectively, p<0.05). The percent signal was significantly reduced to ˜12% after applying the burst sequence, confirming the disruption of intact bubbles that accumulated in the tumor (data not shown). According to the 3D tumor analysis, the PSMA-NB also accumulated more in tumor rim compared to NBs, but the difference was not statistically significant (54.2±4.8%, 38.7±14.4% respectively).

Tumor Extravasation Studies with 3D Ultrasound

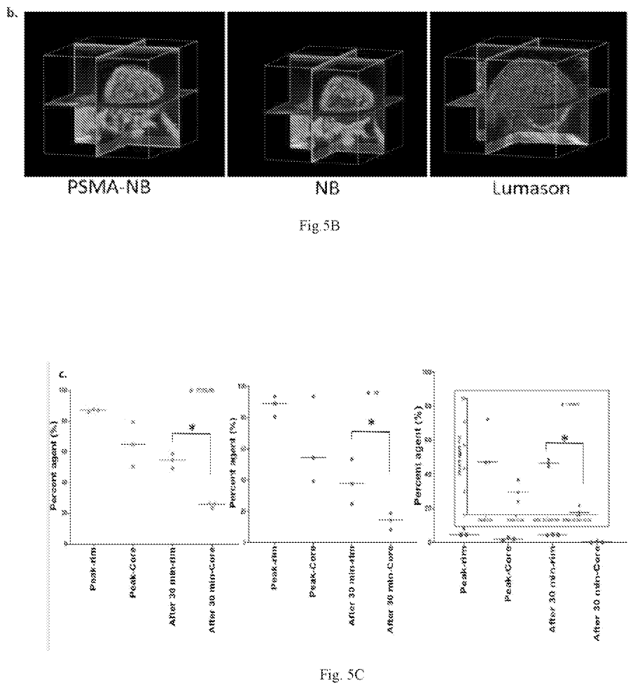

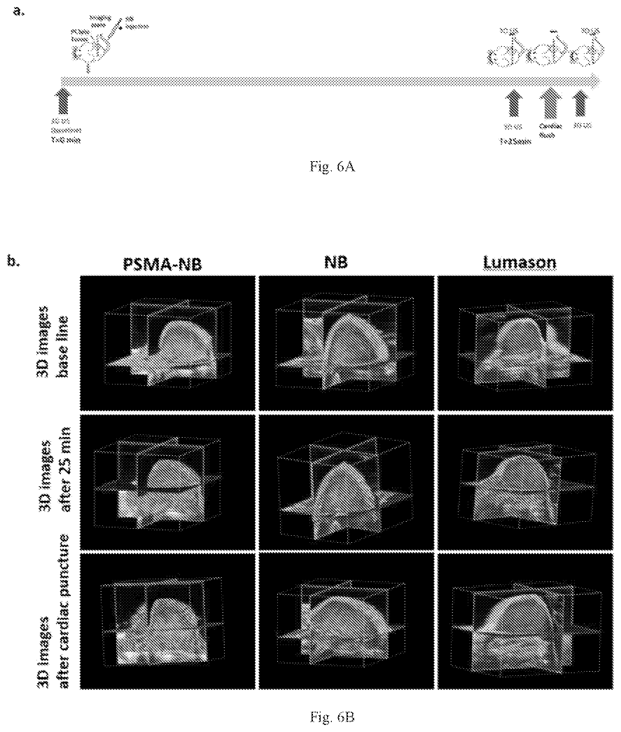

To examine NB extravasation and accumulation in the entire tumor volume in the intact form, whole blood perfusion by cardiac puncture was performed at the 25 min post-injection and the 3D US scan was accomplished before and after cardiac puncture (FIG. 6A). The whole blood perfusion eliminates the blood in the vasculature and removes any material including freely moving nanobubbles from the tumor vasculature. After perfusion, the 3D US signal in the tumor corresponds to the remaining materials in the whole tumor parenchyma or intracellular space. Before perfusion, at 25 min post injection, the percent of PSMA-NB in tumor rim was 1.4-fold higher than that of NB (FIG. 6B, C; 46.8 □21.3 vs 33.2 □25.4%, p=0.2). The percent of PSMA-NB in the tumor core was 4.1-fold higher than that of NB (37.7 □20.1% vs 18.9 □18.7%). After perfusion, the percentage of US signal was reduced in both groups. The PSMA-NB and NB percentages reduced in tumor rim by 67% and 92% respectively, relative to the peak values (15.0 □7.21% vs 2.45 □3.4% respectively). Furthermore, after perfusion, the PSMA-NB was significantly reduced in the tumor core compared to that of the NB (12.2 □2.3% and 3.2 □2.2% respectively (p<0.05). A significantly higher (˜4-fold) 3D US signal for PSMA-NB in tumor core compared to NBs indicates the relatively high accumulation and extravasation of PSMA-NB in the tumor core environment. The presence of US contrast after cardiac perfusion provided evidence that intact NB accumulation and extra avasation in the tumor parenchyma. As expected Lumason percentage was negligibly small at 25 min post injection and after perfusion in both tumor rim and core.

### Histology Analysis

To confirm the US data, histological analysis was proceeded after the Cy5.5-PSMA-NB or CY5.5-NB injected tumor tissues. The PSMA expression, CD31 expression, and the PSMA-NB and NB distribution were evaluated in tumor rim and core separately. The bubbles were tagged with a fluorescent dye; Cy5.5, before the injection. As described in extravasation studies, 25 min of post-injection, 3D US scan was performed and then animal was perfused with PBS by cardiac puncture. After perfusion, rescanned the tumor with 3D as explained above and tumor was harvested for histological analysis. Tumor core and rim was imaged and analyzed separately. The CD31, which represent the vasculature showed higher percentage in tumor rim compared to the tumor core. The PSMA expression was also high in tumor rim compared to tumor core, but no significant different. The Cy5.5-PSMA-NB signal was distributed more evenly in the tumor providing evidence that targeted NB extravasate from the vasculature and accumulated in the tumor matrix. Quantification of histology signal reveal that the Cy5.5-PSMA-NB signal in both tumor core and the rim was significantly higher (3-fold) compared to that of plain NB (p<0.001). (FIG. 7). Enhanced interaction of contrast agent and tumor matrix accounts for the high accumulation of PSMA-NB after extravasation.

### Persistence of PSMA Targeted NB in PC3pip Cells In Vitro

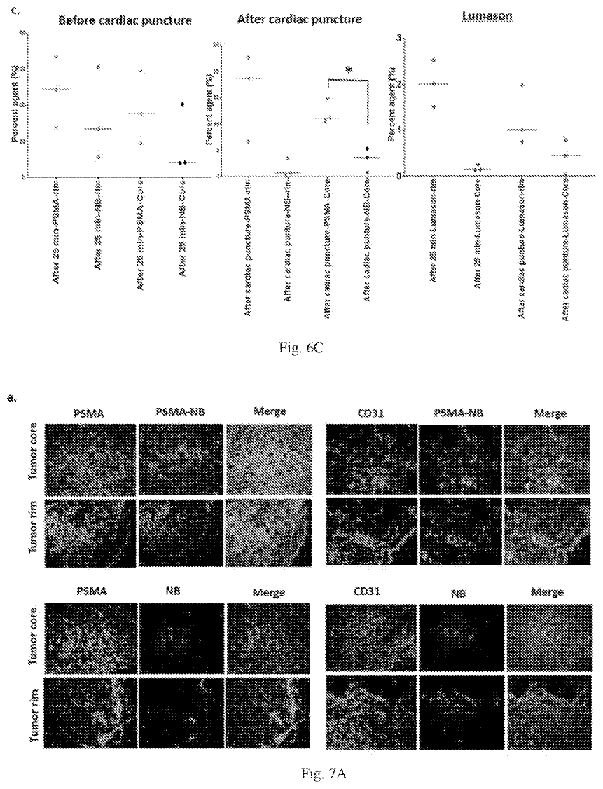

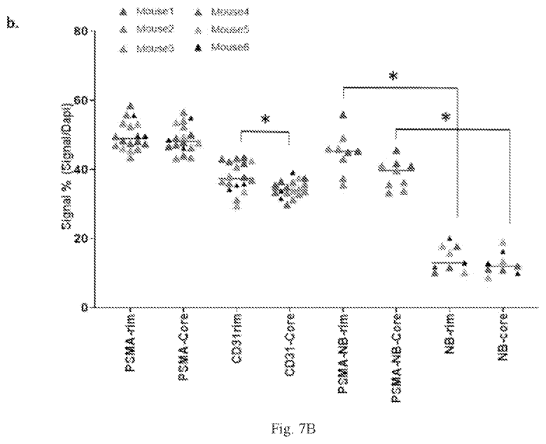

This study investigated the effect of cellular internalization of nanobubble ultrasound contrast agents on the persistence of acoustic activity. Specifically, we compared effects of passive cellular uptake versus receptor-mediated endocytosis of PSMA-targeted NBs in PSMA-expressing human prostate cancer cells. We first investigated the kinetics of nonlinear acoustic properties of both PSMA-positive PC3pip and PSMA-negative PC3flu cells after incubation for 1 h with PSMA targeted or untargeted NBs. As shown in FIG. 8, after incubation for 1 h, PC3pip cells incubated with PSMA-NB showed 3.25 fold higher acoustic activity compared to the plain NB incubated PC3pip cells (FIG. 8; P<0.05; 9.69±1.78 dB vs, 2.19±1.22 dB respectively) and showed higher signal intensity compared to all other groups (NB in PC3pip cells, PSMA-NB in PC3flu cells and plain NB in PC3flu cells) and the negative control (cells only). Furthermore, the significantly higher acoustic activity for internalized PSMA-NBs persisted for all the time points tested except at 48h. (FIG. 8). After 24h, PC3pip cells exposed to PSMA-NBs showed significantly higher US signal intensity (4.11±0.68 dB; P<0.005) compared to all other groups. PSMA-NBs incubated under the same conditions but without cells, showed an initial signal intensity comparable to the PSMA-NB incubated with PC3pip cells. However, after the 3h time point, the signal intensity of PSMA-NB only group was significantly lower than compared to the PSMA-NB internalized in PC3pip cells, indicating the high stability of internalized PSMA-NB in the cellular environment over the free PSMA-NB.

Previous work has examined targeting microbubbles (MB) to a genetically engineered cell surface marker on endothelial progenitor cells (EPC) demonstrated selective binding to EPC in vitro and could be able to image with CEU. It has also been previously shown that either internalized or membrane-bound MB are protected by much greater viscous damping by cells compared to free MB. In agreement with these findings, we also observed that internalized NBs showed significantly higher backscatter for a longer period of time compared to the free NB under the same conditions. Furthermore, at later time points, PSMA-NB in PC3pip cells showed higher contrast than the plain NB incubated with either PC3pip or PC3flu cells and PSMA-NB incubated with the PSMA-negative PC3flu cells. Non-specific uptake of NB by the cells slightly decreases the rate of signal decay from the NBs. However, a much slower decay was observed for the PSMA-NBs localized within the endosomes. Thus, we postulate that the long-time survival of PSMA-NB in cells might be due to stabilization by the endosomal/lysosomal entrapment.

### Confocal Imaging of the PSMA-NB Internalization in PC3pip Cells

PSMA functions as a cell membrane receptor and internalizes the PSMA targeting ligands along with the payload that is attached to the targeting agents. When the PSMA ligand binds to the biomarker on the cell membrane, the cell membrane invaginates and the entire particle is engulfed by the cell. The localization of internalized PSMA-NB within the cells was investigated with confocal microscopic imaging using a fluorescence dye, Lysotracker Red. Lysotracker Red stains late endosomes and lysosomal structures.

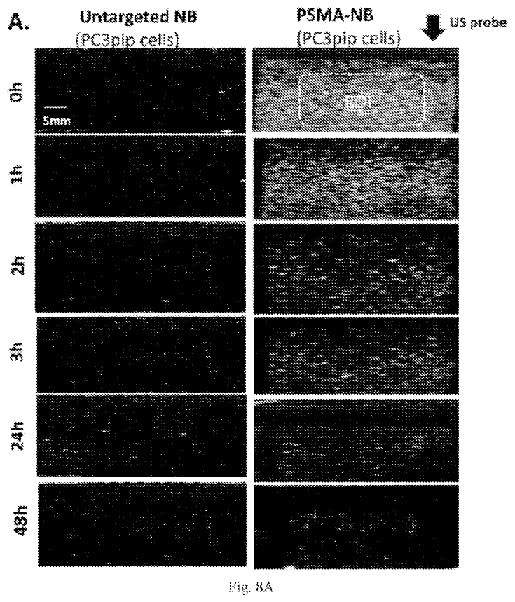

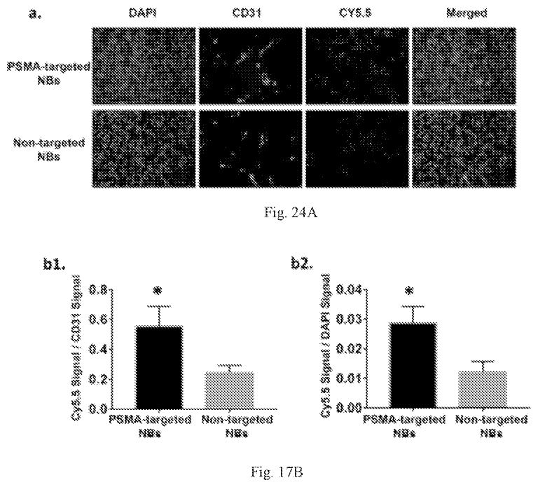

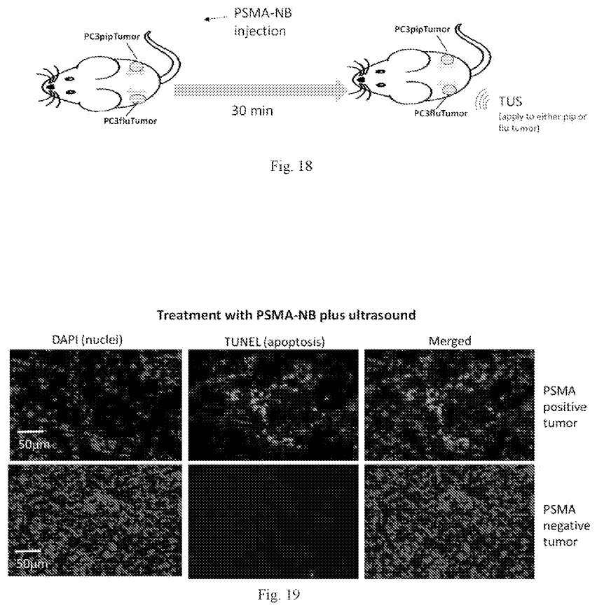

Our previous fluorescence imaging data showed that the PSMA targeted NB are selectively internalized by the PC3pip cells. Confocal imaging results here show internalization, and more specifically, receptor-mediated endocytosis of PSMA-NBs by PC3pip cells (FIG. 9, 100×, FIG. 18, 40×). Substantial co-localization of LysoTracker Red (green) stained late endosomes/lysosomes and Rhodamine-labeled PSMA-NBs (red) was noted in all PSMA-expressing cells. As shown in FIG. 9A, plain NB showed some nonspecific uptake by PC3pip cells but with limited late endosomal/lysosomal co-localization. Uptake of untargeted NBs by PC3pip cells was substantially lower compared to the PSMA-NB uptake. Also, a higher degree of co-localization of PSMA-NB with the late endosomal/lysosomal vesicles was shown in FIGS. 9B and C.

Imaging of PC3pip cells at 24 h after bubble exposure revealed a lower amount of fluorescence signal compared to the earlier time points (FIG. 8D). However, these images also showed a high degree of co-localization of PSMA-NB in late endosomal/lysosomal vesicles. Very low or no fluorescence signal appeared in the cytoplasm or late endosomal/lysosomal compartments of NB incubated cells after 24 h. Furthermore, the images showed yellow color staining near the nucleus, which suggested that the majority of the PSMA-NBs were co-localized with either later endosomes or the lysosomes and transported into cell cytoplasm.

A higher amount of Lysotracker staining was observed when PC3pip cells were incubated with targeted NBs compared to untargeted NBs, which correlates with mechanism of internalization being receptor mediated and entering into the late endosome/lysosome pathway. PSMA has a unique internalization motif and is reported to have a robust baseline internalization rate of 60% of its surface PSMA in 2 hours. Transmembrane location and internalization make PSMA an ideal target for imaging and therapy. Overall, PSMA mediated endocytosis appears to be the main pathway for the internalization of PSMA-targeted nanobubbles in PSMA-expressing prostate cancer PC3pip cells.

Analysis of C₃F₈ in Cells Using Headspace GC/MS

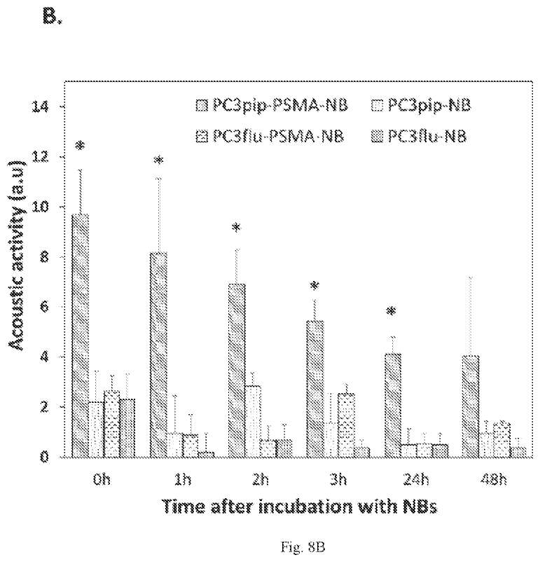

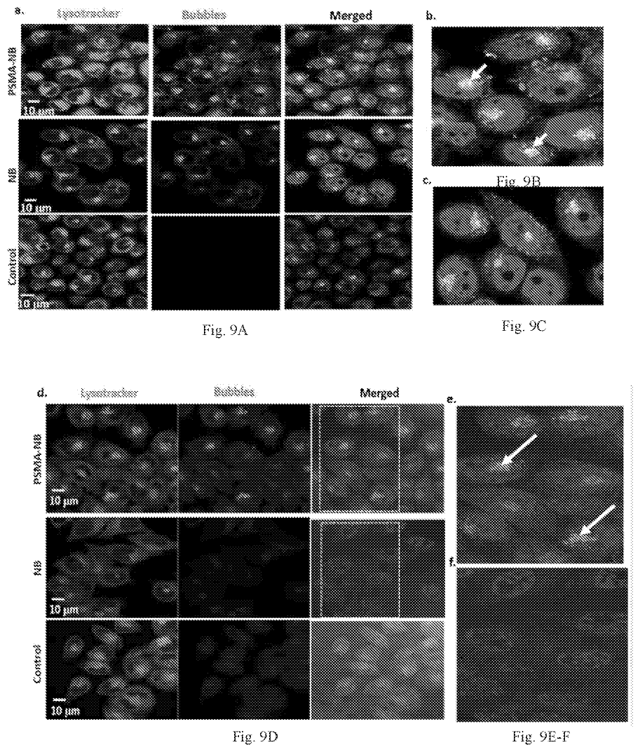

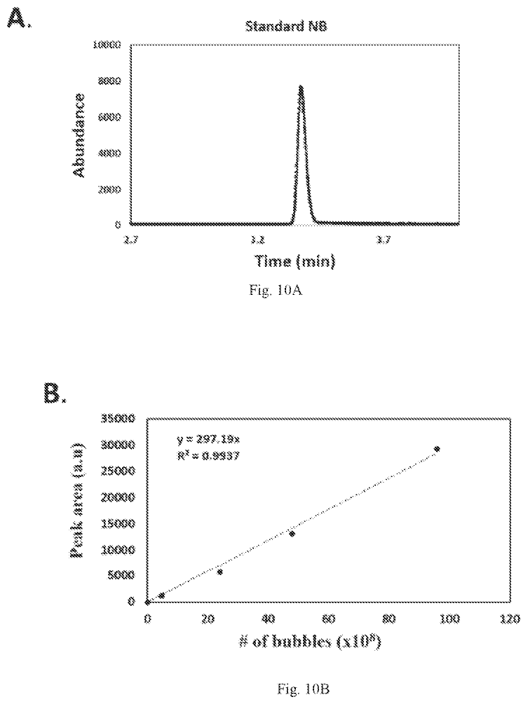

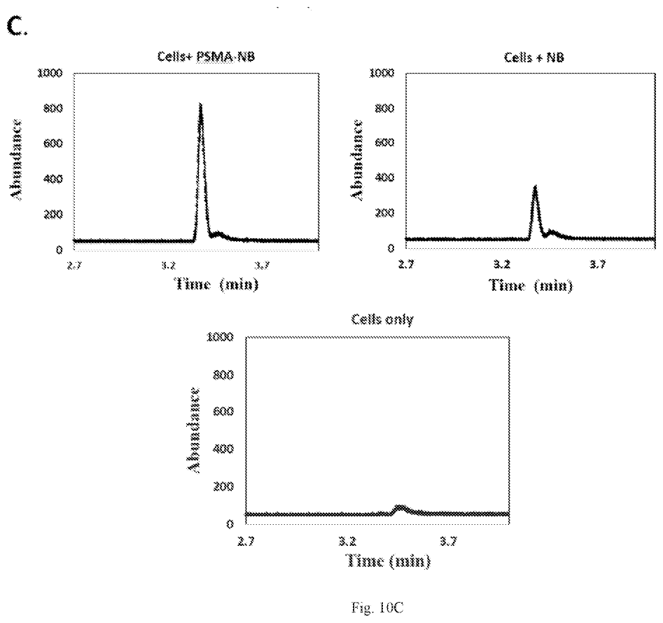

To confirm that intact, gas-bearing nanobubbles internalized into and remain in the PC3pip cells, cells were harvested 3 h following exposure to PSMA-NB or NB, and the presence of C₃F₈ inside the cells was analyzed using headspace GC/MS. The use of headspace GC/MS to quantify C₃F₈ gas concertation in NBs was previously reported and validated. The analysis was performed using the relative abundance of the peak observed at the mass to charge ratio (m/z) of 169, which corresponds to C₃F₈. NBs at different concentrations were used to generate the calibration plot with GC/MS (FIGS. 10A and B). We observed a linear relationship between peak area and the number of bubbles. The GC/MS data showed that the peak area obtained from the PSMA-NB incubated PC3pip cells showed a 3.5-fold higher value compared to that of plain NB incubated PC3pip cell suspension (FIG. 9C; Peak area of PSMA-NB, NB, and cells were 16778 (a.u), 6274 (a.u), and 2172 (a.u) respectively). Based on the calibration curve, this corresponds to approximately 500 average-sized PSMA-NB versus 138 average-size plain NB per cell. To the best of our knowledge, this is the absolute most direct method for confirming the presence of gas vesicles within the cells. The ratio of the peak area is consistent with the difference in acoustic activity from the cells as shown in FIG. 10. It is also strikingly similar to the difference in the ultrasound signal seen from PSMA-NB versus plain NB accumulation in PC3pip tumors after clearance of circulating NBs and supports the data showing that in vivo the targeted NB extravasated and were retained in the tumors in an intact form.

### In Vivo Application of PSMA-NB Internalized Cells

To confirm that the prolonged intracellular retention can also be visualized in vivo, we studied the acoustic activity of internalized bubbles in cells upon injection into mice. PC3pip cells incubated with PSMA-NBs were injected subcutaneously into flank area of nude mice and imaged at 12 MHz. As a control, cells without exposure to NBs were injected adjacent to the labeled cells injected area. FIG. 11A shows the US images obtained 0-24 h and 8 days after cell injection. NB-incubated cells demonstrated significantly high contrast compared to the unlabeled cells immediately after injection (9.7±2.9, 5.2±1.5: P<0.05). The initial signal seen from the control cells is most likely a result of air bubbles entrapped in Matrigel. After 3 and 24 h, the contrast was still significantly higher in regions with NB-incubated cells compared to control cells (FIG. 11B).

Internalized PSMA-NB in PC3pip cells were primarily co-localized with intracellular vesicles and showed substantial backscatter activity for 48 hours after incubation in vitro, and for one week in vivo. To the best of our knowledge, this study shows, for the first time, direct evidence of PSMA targeted-NB uptake and extended retention in cancer cells, and demonstrates the significant role of endosomes/lysosomes in stabilization of the NB acoustic activity.

This example demonstrated the active targeting of NB to PSMA increase the extravasation and the accumulation in PSMA expressing tumor in both 2D non-linear contrast mode and 3D US mode. The data indicated that both tumor wash-in and retention of PSMA-NB are delayed due to biomarker interaction and binding. The longer retention of PSMA-NB signal in tumor core also further supports targeting-driven bubble extravasation. Furthermore, in vitro studies suggested the active targeting of NB to PSMA selectively enhances cellular internalization in PSMA-positive PC3pip cells. US can detect internalized PSMA-NB in PC3pip cells and internalized PSMA-NB showed prolonged stability in the cellular environment, most likely due to entrapment in endosomal vesicles. GC/MS analysis further confirms the intact NB persistence in cells after internalization. The study findings support prior studies showing prolonged acoustic activity of targeted nanobubbles in biomarker expressing tumors and open doors for new molecular imaging and targeted therapy approaches using ultrasound.

### Example 2

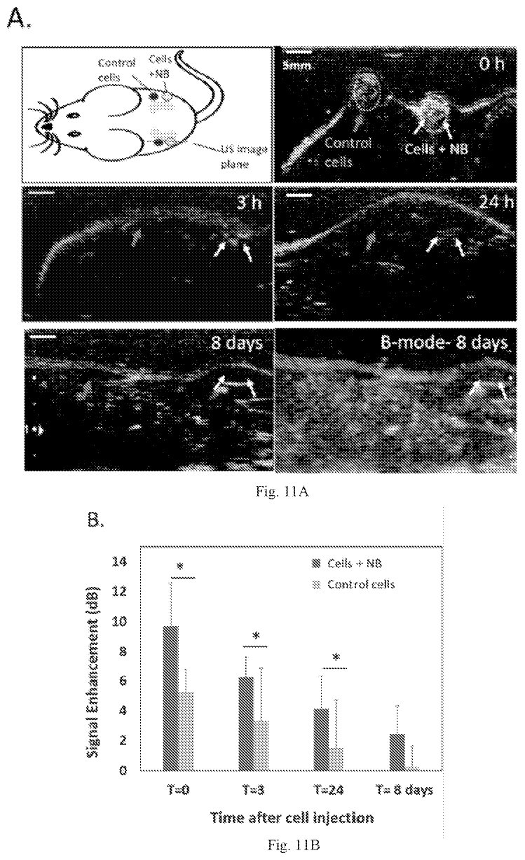

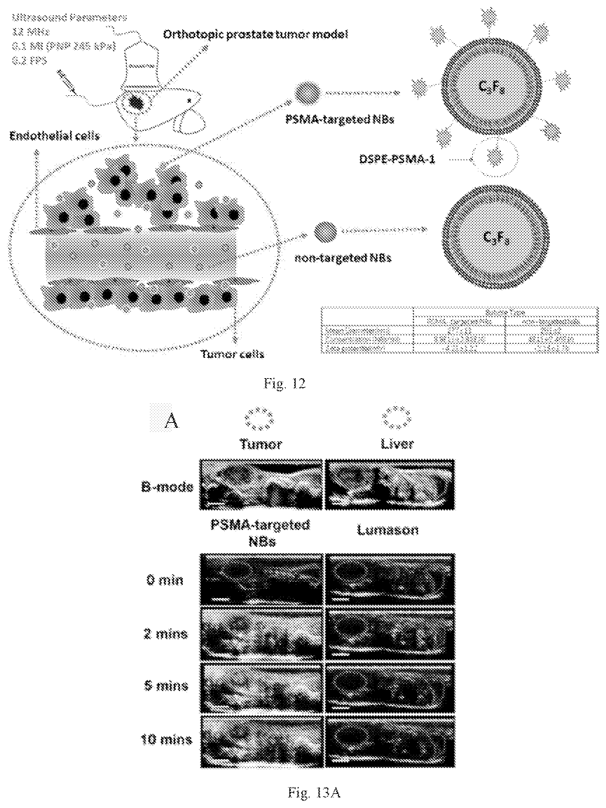

In this example, we investigate PSMA-targeted NBs for US imaging of PCa in vivo using a more clinically relevant orthotopic prostate tumor model in nude mice (FIG. 12). Given the robust nature of the NB-enhanced ultrasound, we also used the technique to examine the effect of PSMA-targeting efficiency on tumor progression and size in the same model. This may provide methods for relevant studies on targeted ultrasound NBs.

### Materials and Methods

### Preparation of PSMA-Targeted and Non-Targeted NB

PSMA-targeted NB (10 mg/mL) was pre-pared as previously reported by first dissolving a mixture of lipids comprising of 1,2-dibehenoyl-sn-glyc-ero-3-phosphocholine (C22, Avanti Polar Lipids Inc., Pelham, Ala.), 1,2 Dipalmitoyl-sn-Glycero-3-Phosphate (DPPA, Corden Pharma, Switzerland), 1,2-dipalmitoyl-sn-glycero-3-phosphoethanolamine (DPPE, Corden Pharma, Switzerland), and 1,2-distearoyl-snglycero-3-phosphoethanolamine-N-[methoxy(polyethylene glycol)-2000] (ammonium salt) (DSPE-mPEG 2000, Laysan Lipids, Arab, Ala.) into propylene glycol (0.1 mL, Sigma Aldrich, Milwaukee, Wis.) by heating and sonicating at 80° C. until all the lipids were dissolved. Mixture of glycerol (0.1 mL, Acros Organics) and phosphate buffered saline (0.8 mL, Gibco, pH 7.4) preheated to 80° C. was added to the lipid solution. The resulting solution was sonicated for 10 min at room temperature. DSPE-mPEG-PSMA (25 μL in 1 mg/mL PBS) was added. The solution was transferred to a 3 mL-headspace vial, capped with a rubber septum and aluminum seal, and sealed with a vial crimper. Air was manually removed with a 30 mL-syringe and was replaced by injecting octafluoropropane (C₃F₈, Electronic Fluorocarbons, LLC, PA) gas. The phospholipid solution was then activated by mechanical shaking with a VialMix shaker (Bristol-Myers Squibb Medical Imaging Inc., N. Billerica, Mass.) for 45 s. PSMA-targeted NBs were isolated from the mixture of foam and microbubbles by centrifugation at 50 rcf for 5 min with the headspace vial inverted, then 200 μL PSMA-targeted NB solution was withdrawn from a fixed distance of 5 mm from the bottom with a 21G needle. Similar preparation was carried out for non-targeted NB but without the addition of DSPE-mPEG-PSMA.

### Size, Concentration, and Surface Charge of NBs

The size distribution and concentration of PSMA-targeted NBs and non-targeted NBs were characterized with a Resonant Mass Measurement (ARCHIMEDES, Malvern Panalytical) equipped with a nanosensor capable of measuring particle size between 50 and 2000 nm. The NB solution was diluted with PBS (500×) to obtain an acceptable limit of detection (<0.01 Hz) and s loaded at 2 psi for 120 s and analyzed at (500×) was measured with an Anton Paar Litesizer 500.

### Animal Models

Animals were handled according to a protocol approved by the Institutional Animal Care and Use Committee (IACUC) at Case Western Reserve University and were in accordance with all applicable protocols and guidelines in regard to animal use. Four to six-week old male athymic Balb/c nude mice were purchased from Case Western Reserve University animal research center and housed in the small animal imaging center, an approved Animal Resource Center. All animals received standard care: Ad libitum access to food and water; 12/12 light/dark cycle; Species appropriate temperature and humidity; Environmental enrichment and group housing whenever possible; Standard cage sanitization; and solid bottom caging. Mice were anesthetized with inhalation of 1-2% isoflurane with 0.5-1 L/min oxygen. A 28½-gauge insulin needle was inserted into ventral prostate gland to deliver 10 μL PSMA (+) PC3pip cells suspended in PBS (phosphate buffered saline). A well-localized bleb within the injected prostate lobe is a sign of a technically satisfactory injection. Animals were observed every other day until tumors reached at about 3-5 mm in diameter, and then used for imaging studies.

### Pharmacokinetic Study

Animals were used in the study 10 days after inoculation when the tumor diameter reached 3-5 mm. The pharmacokinetics of the NBs were monitored by APLIXG SSA-790A Toshiba Medical Imaging Systems (Otawara-Shi, Japan) using the ultrasound probe PLT-1204BT. After mice were anesthetized with 1-2% isoflurane with 0.5-1 L/min oxygen, each mouse was placed in the face-up position, and the ultrasound probe (PLT-1204BT) was placed longitudinally to the axis of the animal body to visualize the ultrasound images of the PC3pip orthotopic tumors. To compare contrast enhanced ultrasound images with the same tumor in the same mouse (n=11), 200 μL of either PSMA-targeted NBs (3.9±0.282×1011/mL) or non-targeted NBs (4.0±0.245×1011/mL) were administrated via tail vein. Before NB injections, the images were acquired in raw data format for 5 s. After injection of NBs, contrast harmonic imaging (CHI) was used to image the change of tissue contrast density (CHI, frequency 12.0 MHz; MI, 0.1; dynamic range, 65 dB; gain, 70 dB; imaging frame rate, 0.2 frames/s). Mice were imaged continuously for 30 min. The remaining NBs were burst by repeated flash replenish and then the same mouse received non-targeted NBs or PSMA-targeted NBs 30 min later. LUMASON (200 μL, 1-5×108/mL, sulfur hexafluoride lipid-type A microspheres, Bracco Diagnostics Inc.) was tested in the other 3 mice. LUMASON was prepared according to the protocol provided by the manufacturer. The raw data were processed with software provided by the scanner manufacturer. The acquired linear raw data images were processed with CHI-Q quantification software (Toshiba Medical Imaging Systems, Otawara-Shi, Japan). Regions of interest (ROIs) were drawn outlining the areas of the tumor and the liver. The signal intensity in each ROI as a function of time (time-intensity curve—TIC) was calculated and exported to Excel. To analyze the decay of ultrasound contrast, the baseline was subtracted from TIC.

### Bubble Burst Study

Mice received 200 μL of NBs (3.9±0.282×1011/mL) via tail vein injection. Five minutes after contrast agent injection, images were taken in 4 different planes including tumor and liver in the same field of view, and then 25-times flashing were used in different positions from the liver plane to the heart plane in order to burst all the NBs left in the circulation. After that, images were taken again in 4 different planes including the tumor and the liver in the same field of view using contrast-mode imaging. The average intensity was analyzed by Image J. The experiment was repeated in 4 nude mice bearing PC3pip orthotopic tumors.

### Histological Analysis

Animals were divided into 3 groups: PSMA-NB (n=3), plain-NB (n=3), and no contrast control (n=3). The method was the same as our previous study. Mice received either 200 μL of contrast material or PBS alone via tail vein. Ten minutes after contrast agent injection, PBS perfusion was performed with 50 mL PBS though left ventricle. After perfusion tumors and livers were harvested and embedded in optimal cutting temperature compound (OCT Sakura Finetek USA Inc., Torrance, Calif.). The tissues were cut into 9 μm slices, and then CD31 staining was performed to visualize the tumor vessels. Briefly, tissues were washed 3 times with PBS and incubated with protein blocking solution that contain 0.5% Triton X-100 (Fisher Scientific, Hampton, N.H.). Then tissues were incubated in 1:250 diluted CD31 primary antibody (Fisher Scientific, Hampton, N.H.) for 24 h at 4° C. After washed with PBS, tissues incubated with Alexa 568 tagged secondary antibody (Fisher Scientific, Hampton, N.H.) for one hour and stained with DAPI (Vecor Laboratories, Burlingame, Calif.) using standard techniques. Then fluorescence images were observed under Leica DM4000B fluorescence microscopy (Leica Microsystem Inc, Buffalo Grove, Ill.) and then analyzed by Image J

### Results

### Contrast-Enhanced Ultrasound Imaging of Orthotopic Prostate Tumors Using PSMA-Targeted NBs and LUMASON

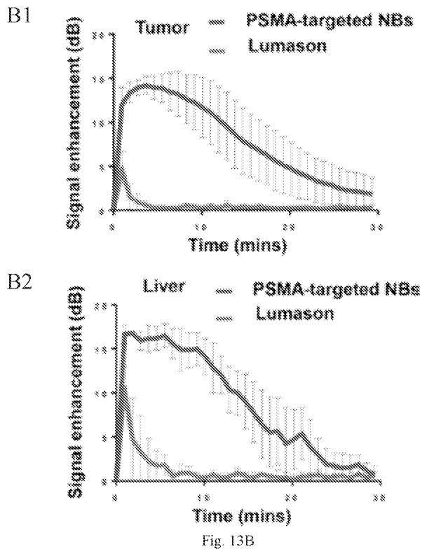

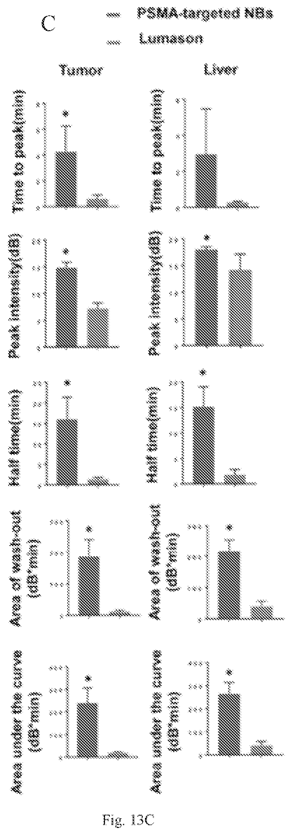

After tail vein injection of PSMA-targeted NBs (200 μL of 3.9±0.282×1011/mL PSMA-targeted NBs) (n=11) or LUMASON (200 μL of 1-5×108/mL LUMASON MBs) (n=3), contrast har-monic imaging (CHI) images were continuously acquired (receive frequency of 12 MHz) to determine the dynamics of the bubbles in the tumors and livers. The LUMASON dose, PSMA-targeted NB dose and imaging parameters used were optimized in our previous work. It is worth noting that the nonlinear contrast imaging parameters in these studies utilized a higher frequency than typically used clinically for LUMASON (3 MHz). While these should not affect the kinetic parameters of LUMASON, they may affect the overall image quality. Under CHI mode, tumors and livers were not visible before injection of either PSMA-targeted NBs or LUMA-SON (FIG. 13A). A rapid enhancement started approximately 15-30 s after NB injection, and was observed first in the livers followed by tumors. The UCA kinetic parameters (FIG. 13C) were obtained from the time intensity curve (TIC) (FIG. 13B1,B2). These include time to peak, peak intensity, half time, area of wash-out and area under the curve (AUC). These parameters were compared between PSMA-targeted NBs and LUMASON both in the tumor and the liver. Although the group size for the LUMASON group was relatively small, the difference between the LUMASON group and PSMA-targeted NB group at the imaging parameters used in this study was large and a statistically significant difference was observed between the two groups. This is also consistent with previously published work. The results showed that the time to peak, peak intensity, half time, area of wash-out and AUC were significantly different between PSMA-targeted NBs and LUMASON (p<0.05) in the tumor, and the last four parameters were significantly different between PSMA-targeted NBs and LUMASON (p<0.05) in the liver. All above indicated higher stability and longer circulation time of our PSMA-targeted NBs than for LUMASON MBs in the blood stream.

The tumor sizes in LUMASON group were between 280 and 520 mm³ and the tumor sizes in NB groups were from 90 to 1100 mm³. To make sure that the difference between LUMASON and PSMA-targeted NB was not a result of the tumor size, we split the PSMA-targeted NB groups into two groups based on tumor size: Group A (small tumor) had tumor volumes between 90 and 670 mm3 and Group B (big tumor) had tumor volumes between 670 and 1100 mm3 and compared the LUMASON group to these two groups separately. The parameters in both group A and Group B were nonetheless significantly different from those in the LUMASON group. Our results confirmed that the lower peak enhancement of LUMASON was not related to the tumor size.

### Contrast Agent Dynamics and Comparison of Orthotopic Prostate Tumors Using PSMA-Targeted NBs and Non-Targeted NBs

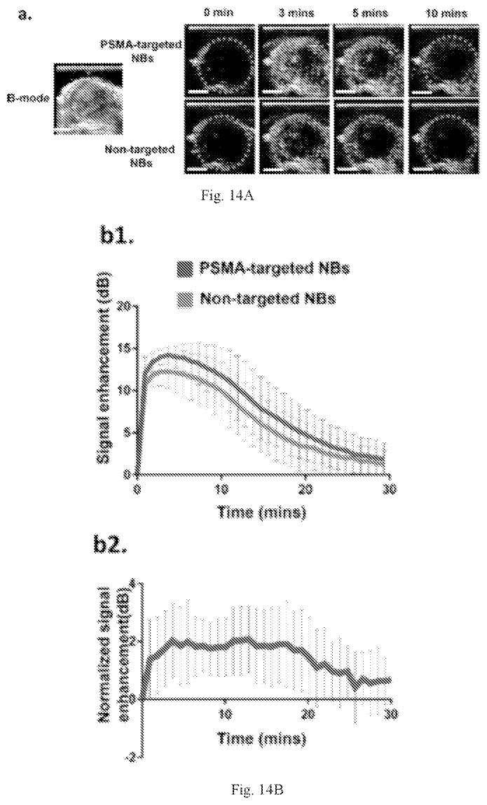

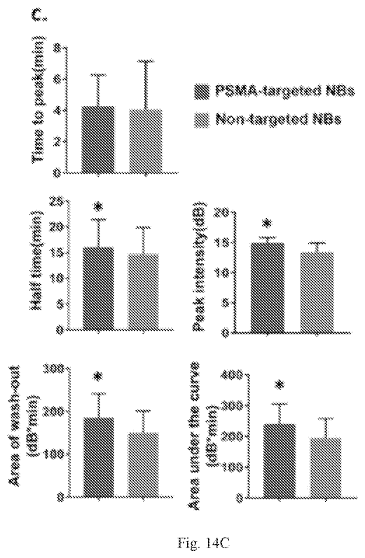

To evaluate the selective imaging ability of PSMA-targeted NBs toward prostate tumor, non-targeted NBs were used as a comparison. US scans with both bubble formulations were performed under identical conditions, and the average results of 11 nude mice bearing PC3pip orthotopic tumors were reported. First, the PC3pip tumors were localized in B-mode, and then we switched to contrast mode. Tumors were not visible in the contrast mode before bubble injection (FIG. 14A). Continuous contrast mode US was performed to monitor the bubble dynamic in the tumors after i.v. injection of PSMA-targeted NBs or non-targeted NBs. The bubble kinetics obtained from the time intensity curve (TIC) (FIG. 14B1), which includes time to peak, peak intensity, half time, area of wash-out and area under the curve (AUC), were compared among PSMA-targeted NBs and non-targeted NBs in the PSMA (+) PC3pip orthotopic tumors (FIG. 14C). Significant differences in peak intensity (p=0.0001), half time (p=0.0056), area of wash-out (p=0.0092) and area under the curve (p<0.0001) were measured between PSMA-targeted NBs and non-targeted NBs. US signal obtained from non-targeted NBs measurements was used to normalize the signal from PSMA-targeted NBs. The normalized TIC showed that the average intensity from PSMA-targeted NBs was always higher than non-targeted NBs at different time points (FIG. 14B2). Since the tumor sizes used in this study varied from 90 to 1100 mm³, we also divided the animals into two cohorts: Group A (small tumor, 90-670 mm³, n=7) and Group B (big tumor, 670-1100 mm³, n=4) and compared the parameters of PSMA-targeted and non-targeted NBs. In Group A with small tumors, significant differences in peak intensity and area under the curve were observed. In Group B with big tumors, significant differences in peak intensity, area under the curve and half time were seen. The TIC of individual mice also showed differences between PSMA-targeted NBs and non-targeted NB in 10 out of 11 mice. Although inter-animal viability was observed, the overall results between the two groups were similar. Altogether, our data indicated higher stability and longer circulation time of our PSMA-targeted NBs than that of non-targeted NBs in the blood stream.

### Contrast Agent Dynamics and Comparison Based on Different Tumor Sizes

Due to apparent variability in the dynamics of PSMA-targeted NBs and non-targeted NBs depending on the size of the tumor, tumors were separated into two groups: Group A had tumor volumes between 90 and 670 mm³ (n=7), and Group B had tumor volumes between 670 and 1100 mm³ (n=4). The UCA kinetic parameters (FIG. 15B) were obtained from the time intensity curve (TIC) (FIG. 15A). As tumor sizes increased, the peak intensity, area of wash-out, and total area under the curve were significantly different between Group A and Group B (FIG. 15B). As shown in FIG. 14A, some part of the tumor did not fill well after bubble injection in Group B, and the signal was heterogeneous in B-mode; while in Group A, the signal was relatively homogeneous both in B-mode and contrast mode (FIG. 13A).

PSMA-Targeted NBs are Retained in the Orthotopic Prostate Tumor after Bubble Clearance from Circulation

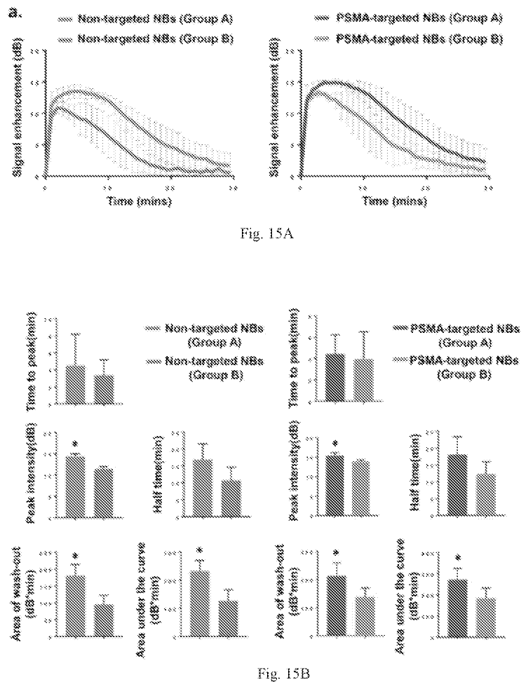

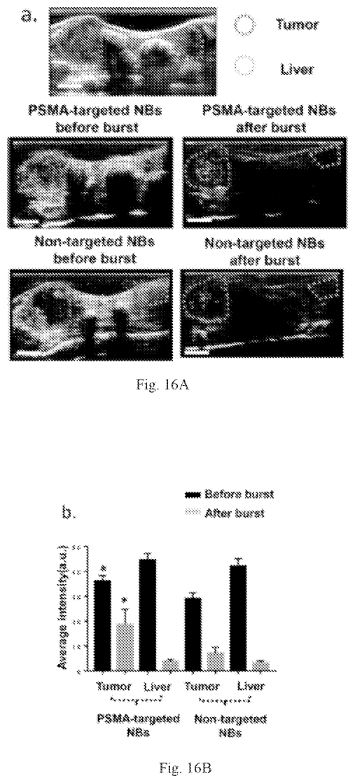

The bubble burst studies were performed in 4 additional mice bearing orthotopic PC3pip tumor at the size of 300-800 mm3 and the average results were reported in FIG. 16. The series of images in FIG. 16A showed the CHI images before and after bursting the bubbles in circulation via repeated high intensity pulses applied to the liver. Quantitative analysis of the enhancement (FIG. 16B) showed a 47.9±18.6% reduction in signal after clearance in the PC3pip tumors with targeted NBs, compared to 74.8±8.9% with non-targeted NBs in tumor and 92.2±2.4% in the liver. These data showed significantly higher peak enhancement in the tumors enhanced using PSMA-targeted NBs compared to non-targeted NBs. Most importantly, following the clearance of circulating NBs via related hi-intensity pulses, the signal intensity in tumors enhanced using PSMA-targeted NBs remained significantly higher compared to non-targeted NBs. This suggests significant NB retention in PSMA-expressing PCa cells. In contrast to the tumors, the signal intensity in the liver was similar with both PSMA-targeted NBs and non-targeted NBs before the burst and was nearly completely eliminated after clearance for both agents. It is important to note that the tumor region in these studies was not exposed to constant insonation (in contrast with the tumors used to generate the TIC) which preserved bubble echogenicity for a longer time. This is likely to have magnified the differences between targeted and untargeted NB s seen in this experiment. This data shows, for the first time, significant extravascular retention of PSMA-targeted NBs in PSMA-positive PC3pip tumor parenchyma (likely within the tumor cells) after clearance of NBs from circulation in live mice.

### Immunohistochemical Analysis

To further validate that PSMA-targeted NBs could extravasate into the tumor matrix, the bubbles were labeled with a fluorescent dye Cy5.5 and injected to a new set of animals bearing orthotopic PC3pip tumor. Ten minutes after injection, mice underwent a cardiac flush perfusion procedure with cold PBS to remove circulating bubbles and tumors were harvested for histological analysis. CD31 staining was used to visualize the tumor vessels. The fluorescence in the vessels and cells was used to normalize the bubbles signal per field. Histological images showed that Cy5.5 signal of PSMA-targeted NBs group was found outside of tumor capillaries and deep in the parenchyma (FIG. 17A), which provided strong evidence of bubble extravasation and subsequent interstitial penetration. The NB fluorescence ratio (quantification of fluorescence ratio from total bubbles fluorescence/vessels fluorescence and total bubbles fluorescence/cells fluorescence per field) in PSMA-targeted NBs group was significantly higher than that in non-targeted NBs group (FIG. 24B1,B2), which confirmed that PSMA-targeted NBs not only can extravasate into the tumor but also can be trapped within the tumor.

The goal of this example was to formulate a novel targeted, nanoscale ultrasound contrast agent to detect PSMA (+) PCa in a clinically relevant orthotopic model. Our previous study in a flank tumor model has already examined the kinetics of PSMA-targeted NBs and non-targeted NBs, and histological findings confirmed that PSMA-targeted NBs can specifically recognize the tumors with PSMA expression. In this example, significant differences were observed in peak intensity, half time, area of wash-out and area under the curve between PSMA-targeted NBs and non-targeted NBs for orthotopic tumors (FIG. 14). Comparing the results obtained from orthotopic the tumor model to previous work in the flank tumor model, the signal difference between PSMA-targeted NBs and non-targeted NBs in orthotopic PC3pip tumor was less obvious than that in flank PC3pip tumor. More specifically, while the total AUC was comparable for PSMA-NBs between the two models, the untargeted NB AUC and especially the washout AUC were increased in the orthotopic model. West-ern blot studies showed that flank PC3pip tumor and orthotopic PC3pip had similar level of PSMA expression, therefore, the difference was not due to different levels of PSMA expression. We hypothesize that this difference may be a consequence of three factors: 1) variability in vascular density and vascular permeability in the two tumor models, as well as 2) the overall tumor burden and 3) differences in cellular density and central necrosis. Differences between tumor microenvironments between flank and orthotopic tumors are known to affect these factors. Specifically, it has been reported that orthotopic PC3 tumors have higher vascular volume and permeability than flank PC3 tumors. The higher vascular permeability of the orthotopic tumor may enable greater extravasation of all nanobubbles by the enhanced permeability and retention (EPR) effect. It is thus possible that the enhanced permeability will lead to increased uptake of both PSMA-targeted NBs and non-targeted NB; therefore, the difference of NB accumulation in orthotopic tumors between PSMA-targeted NBs and non-targeted NBs accumulation is smaller than that in flank tumor models. This is also reflected by the higher overall area under the time intensity curve (193.7±64.38 dB*min in orthotopic model vs 130.4±50.11 dB*min in flank model) and the washout AUC for the non-targeted NBs (149.5±52.19 dB*min in orthotopic model vs 116.51±25.61bB*min in flank model), which is seen in this model versus the flank tumors. If the necrosis and cellular density are higher, this would also result in greater retention of all bubbles, thus reducing the washout of untargeted ones. In general, there are many potential differences between these models, that can result in the specific changes in the TIC. This is partially why using NB contrast enhanced ultrasound may provide some insight into nanoparticle transport in tumors. The average tumor size in the flank tumor was around 125 mm³, while the average tumor size in the orthotopic tumor was around 500 mm³. Stratifying tumors into large and small cohorts illustrated significant difference in peak intensity, area of wash out and area under the curve (FIG. 15), indicating that tumor burden is also a factor that affects the kinetics. Inter-animal variability was also observed in animals. When normalized to each individual animal (as shown in FIG. 14B2) the difference in enhancement is higher. It is likely that orthotopic tumors are more heterogeneous than flank tumors, thus leading to reduced differences on average.

In this example, a bubble burst study was used to detect the signal in tumor after bursting the circulating bubbles, which indicated that PSMA-targeted NBs were retained in the tumor to a greater extent than non-targeted NBs (FIG. 16). In addition, we also found that kinetics and tumor distribution of our NBs varied depending on tumor size/stage. Histology studies of the small tumors and big tumors found that the center of the big tumors was more necrotic than that of the small tumor.

The targeting ligand, PSMA-1, is a peptide-based highly negatively charged PSMA ligand, which can be used in clinical research and also can be easily synthesized. The average diameter of our PSMA-targeted NBs was 277±11 nm. The smaller size of our NBs should achieve better tumor penetration than bigger size bubbles. Smaller size of particles has been shown to improve the biodistribution and the enhanced permeability and retention effect of nanoparticles in a murine xenograft tumor model. Overall, the current data suggest that: 1) echogenic nanobubbles labeled with a high affinity ligand to PSMA are considerably more stable in vivo and show greater differences in kinetics between clinical MBs and non-targeted NBs; 2) the NBs appear to have distinct kinetics and retention in tumors of different sizes. This could be a promising area of future investigation, as a means of staging and potentially grading tumors using the same agents.

### Example 3

This Example shows the precise tuning of membrane and/or shell composition, nanobubble size and acoustic pulse sequences can elicit superior nanobubble behavior at a given ultrasound frequency and pressure. It was found that the acoustic response of nanobubbles with a narrow size distribution range can be altered by their membrane shell structure (FIG. 2). The membrane composition was modulated by the inclusion of glycerol and propylene glycol (PG). PG has been shown to increase lipid membrane fluidity, while glycerol leads to stiffening of the shell. To conduct this experiment, NBs with a C₃F₈ gas core were formulated as previously described. Briefly, a cocktail of lipids including DBPC, DSPE-PEG2000 and glycerol were dissolved in PG and PBS followed by gas exchange and activation via mechanical agitation. NBs with low polydispersity were prepared via isolation by centrifugation, and filtration through a 400-nm membrane. The shell- and pressure-dependence of the onset of detectable nonlinear response of NB solutions in PBS were determined using US in contrast harmonic mode (Toshiba Aplio XG, 12 MHz) at pressures between 74 kPa and 857 kPa (MI=0.03 to 0.35). As shown in FIG. 2, bubbles with a more flexible shell compromised of PG plus a cocktail of phospholipids showed a significantly lower pressure needed for the start of activity (as measured by the signal intensity in the imaging field of view). These bubbles showed an onset of detectable nonlinear response at lower pressures (123 kPa to 245 kPa). US images of stiff NB dispersions showed a minimal 6% signal increase for pressure increasing from 343 kPa to 465 kPa and a considerable 146% increase from 465 kPa to 710 kPa.

The controllable pressure threshold has potential advantages to contrast enhanced methods based on the nonlinear response of bubbles. One of these techniques is amplitude modulation where two pulses with different pressure amplitude is sent to tissue. One pulse usually has the amplitude of twice the other pulse. Signals are scaled and subtracted upon receive. Due to the linear response of the tissue, the signal from tissue cancels and the only remaining signal is from bubbles. Thus, contrast to tissue (CTR) increases. Sending a pulse below the pressure threshold and sending one above the threshold for enhancement will significantly increase the CTR. The applications of flexible shells result in a smaller pressure for the enhancement which leads to a higher scattering cross section and thus better outcome for imaging purposes. Applications of stiffer shells, skew the pressure to higher values, thus making them more suitable for therapeutic purposes like enhanced heating applications where higher pressures are required. Moreover, due to the negligible oscillation amplitude of the pre-focal NBs and taking advantage of the steep pressure gradients of some ultrasound transducers we can significantly decrease the attenuation of the pre-focal bubbles. Thus, delivering sufficient energy to the resonant NBs at the target which will contribute more efficiently to the enhanced heating effects. Moreover, undesired heating in the off-target region is minimized due to the off resonant bubbles. Finally, it is also expected that this type of approach will be more effective in eliciting antitumoral-induced immunity through the so-called “abscopal effect”, reported for high intensity focused ultrasound and histotripsy.

In summary, the TNT application bubbles with specific shell compositions that 1) lower the general activation pressure, and 2) exhibit tunable and predicable pressure-sensitive behavior are necessary and 3) enable cell-mediated endocytosis and prolonged residence in intracellular vesicles. This is what makes the technology unique compared to traditional microbubble-mediated cell disruptions.

### Example 4

This example shows the results of in vitro cellular uptake studies and in vivo experiments demonstrating that targeting NB with PSMA-1 ligand selectively increased the binding to PSMA-expressing PC3pip cells and high accumulation in PC3pip tumor. We hypothesize that accumulated PSMA targeted NB combined with therapeutic US selectively damage the PSMA positive PC3pip tumor tissues via intracellular explosion.

### Preparation and Characterization NBs

Lipid solution (10 mg/mL) for nanobubbles was prepared by dissolving 1,2-dibehenoyl-sn-glycero-3-phosphocholine (DBPC, Avanti Polar Lipids Inc., Pelham, Ala.), 1,2-Dipalmitoyl-sn-glycero-3-Phosphate; DPPA, 1,2-dipalmitoyl-sn-glycero-3-phosphor ethanolamine; DPPE (Corden Pharma, Switzerland), and 1,2-distearoyl-sn-glycero-3-phosphoethanolamine-N-[methoxy (polyethylene glycol)-2000] (ammonium salt) (DSPE-mPEG 2000, Laysan Lipids, Arab, Ala.) with 6:1:2:1 ratio in propylene glycol (PG, Sigma Aldrich, Milwaukee, Wis.) by heating and sonicating at 80° C. Mixture of glycerol (Gly, Acros Organics) and phosphate buffer solution (0.8 mL, Gibco, pH 7.4) preheated to 80° C. was added and sonicated for 10 min at room temperature. The solution (1 mL) was transferred to a 3 mL headspace vial, capped with a rubber septum and aluminum seal. Air was replaced by octafluoropropane (C3F8, Electronic Fluorocarbons, LLC, PA) gas and activated by mechanical shaking with a VialMix shaker (Bristol-Myers Squibb Medical Imaging Inc., N. Billerica, Mass.) for 45 s. Nanobubbles were isolated from the microbubbles by centrifugation at 50 rcf for 5 min with the headspace vial inverted, and the 100 μL NB solution withdrawn from a fixed distance of 5 mm from the bottom with a 21G needle.

PSMA-NB were prepared by adding DSPE-PEG-PSMA-1 (25 μg/ml) to the initial lipid solution and followed the above protocol. To prepare DSPE-PEG-PSMA-1, PSMA-1 (from prof. James Basilion lab) was mixed with DSPE-PEG-MAL (1,2-distearoyl-sn-glycero-3-phospho ethanolamine-N-[methoxy (polyethylene glycol)-2000-Maleimide, Laysan Bio, Arab, Ala.) in 1:2 ratio at pH 8.0 in PBS. After combined, the mixture was vortexed thoroughly and was reacted for 4h on the vial rotator at 4° C. The product was lyophilized and the resultant powder was dissolved in PBS to obtain DSPE-PEG-PSMA-1 stock solution. Conjugation of DSPE-PEG-PSMA-1 was confirmed by High Performance Liquid Chromatography (HPLC) and MALDI TOF technique. HPLC was performed on a Shimadzu HPLC system equipped with a SPD-20A prominence UV/visible detector and monitored at a wavelength at 220 nm. Analytical HPLC was performed using an analytical Luna 5μ, C18(2) 100A column (250 mm×4.6 mm×5 μm, Phenomenex) at a flow rate of 1.0 mL/min. Gradient used was 10%-40% Acetonitirle against 0.1% TFA over 20 min.

The size distribution and concentration of NBs were characterized with resonant mass measurement (Archimedes®, Malvern Panalytical) as explained earlier1-2. Measurement was finalized after 1000 particles were measured. Data was exported from the Archimedes software (version 1.2) and analyzed for positive and negative counts1. Surface charge of the diluted NB solution (500×) was measure with an Anton Paar Litesizer 500.

### Procedure

FIG. 18 illustrates a schematic of a procedure for administering and insonating PSMA-NBs to mice. The procedure includes:

Inject dual tumor mice with 200 ul of targeted PSMA-NB to mice M1, M2 and M3, M4) via tail vein.

After 30 min for PC3pip tumor of M1 and M3 mice apply TUS.

Also, after 30 min for PC3flu tumor of M2 and M4 mice apply TUS.

For the control mouse inject PBS and after 30 min for both PC3pip and PC3flu tumor apply TUS. Parameters: TUS treatment; 3 MHz, 2.2 W/cm²/10 DC for 5 min (small probe).

After 24h of the treatment excise the tumor and proceed for histology.

Analyze the apoptosis with TUNEL assay.

Group 1—PSMA-NB injection and after 30 min TUS application for PC3pip tumor (M1 and M3)

Group 2—PSMA-NB injection and after 30 min TUS application for PC3flu tumor (M2 and M4)

Group 3—PBS injection and after 30 min TUS application for PC3pip and PC3flu tumor (M5)

FIG. 19 shows that when PSMA-expressing tumors were treated with PSMA-targeted nanobubbles in combination with ultrasound at 10% duty cycle, 3 MHz, 2.2 W/cm² for 5 minutes, significant apoptosis was seen at 24 h after treatment. Little to no apoptosis was seen when the same treatment was applied to PSMA-negative tumors. This suggests that only those nanobubbles that were internalized into tumor cells via receptor-mediated endocytosis had a significant toxic effect.

FIG. 20 shows that when PSMA positive or PSMA negative tumor were treated with ultrasound only (no bubbles) at 10% duty cycle, 3 MHz, for 5 minutes, little to no apoptosis was seen. This suggests that only nanobubbles in combination with ultrasound have a toxic effect. No effect was seen also with bubbles alone (no ultrasound). This data is not shown.

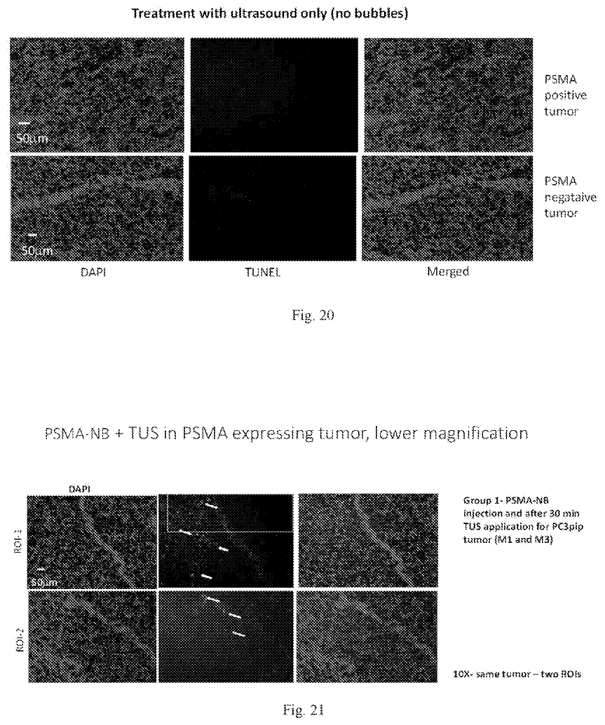

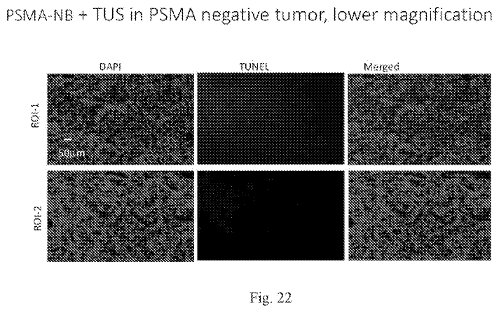

FIGS. 21 and 22 show the tumors which express PSMA treated with PSMA-NB and TUS showed significant apoptosis in cells death compared to all the other groups. The apoptosis is approximately 33 (33.55±1.10) % of the DAPI for the ROI selection.

From the above description of the invention, those skilled in the art will perceive improvements, changes, and modifications. Such improvements, changes, and modifications within the skill of the art are intended to be covered by the appended claims. All references, publications, and patents cited in the present application are herein incorporated by reference in their entirety.

Having described the invention, we claim:

1. A method of inducing cell death in a subject, the method comprising:
administering to the subject a plurality of nanobubbles, each nanobubble having a membrane that defines at least one internal void, which includes at least one gas, and a targeting moiety that is linked to an external surface of the membrane, wherein the target moiety binds to a cell surface molecule of a target cell and wherein the nanobubble has a size and/or diameter that facilitates internalization of the nanobubble by the target cell upon binding of the targeting moiety to the cell surface molecule; and
insonating nanobubbles internalized into the target cell with ultrasound energy effective to promote inertial cavitation of the internalized nanobubbles and apoptosis and/or necrosis of the target cell.

2. The method of claim 1, wherein the nanobubbles have an average diameter of about 50 nm to about 400 nm.

3. The method of claim 1, wherein the cell is a cancer cell and the targeting moiety binds a cancer cell surface molecule.

4. The method of claim 1, wherein the targeting moiety is selected from the group consisting of polypeptides, polynucleotides, small molecules, elemental compounds, antibodies, and antibody fragments.

5. The method of claim 1, wherein the cancer cell surface molecule is a cancer cell antigen on the surface of a cancer cell.

6. The method of claim 4, wherein the cancer cell antigen comprises at least one of 5T4, α2β1 integrin, AXL receptor tyrosine kinase (AXL), B-cell maturation antigen (BCMA), c-MET (Hepatocyte Growth Factor Receptor), C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, collagen receptor, Cripto protein, CS1, delta-like canonical Notch ligand 3 (DLL3), endothelin receptor type B (EDNRB), ephrin A4 (EFNA4), epidermal growth factor receptor (EGFR), EGFRvIll, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), glycoprotein non-metastatic B (GPNMB), guanylate cyclase 2 C (GUCY2C), human epidermal growth factor receptor 2 (HER2), human epidermal growth factor receptor 3 (HERS), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), protein tyrosine phosphatase mu (PTPmu) solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), or trophoblast cell-surface antigen (TROP-2).

7. The method of claim 1, wherein the targeted cell is a prostate cancer cell and the cell surface molecule is PSMA.

8. The method of claim 1, wherein the membrane is a lipid membrane

9. The method of claim 8, wherein the lipid membrane further includes at least one of glycerol, propylene glycol, pluronic (poloxamer), alcohols or cholesterols, that change the modulus and/or interfacial tension of the bubble membrane.

10. The method of claim 8, wherein the nanobubbles have a lipid concentration of at least about 5 mg/ml.

11. The method of claim 8, wherein the lipid membrane includes a mixture of at least two of dipalmitoylphosphatidylcholine (DPPC), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA), or PEG functionalized lipids thereof.

12. The method of claim 11, wherein the mixture of lipids includes at least about 50% by weight of dibehenoylglycerophosphocoline (DBPC) and less than about 50% by weight of a combination of additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof.

13. The method of claim 1, wherein gas comprises a perfluorocarbon gas.

14. The method of claim 1, wherein the insonation induces cell death without adversely effecting normal cells and tissues.

15. The method of claim 1, wherein the insonation is at a duty cycle of about 1% to about 50%, an ultrasound frequency of about 1 MHz to about 12 MHz, an intensity of about 0.1 W/cm2 to about 3 W/cm2, a pressure amplitude of about 50 kPa to about 1 MPa, and a time of about 1 minute to about 10 minutes.

16. The method of claim 1, wherein the insonation comprises two ultrasound pulse sequences with pulses of different pressure amplitudes sent to tissue in which the nanobubbles are administered, wherein one pulse has a pressure amplitude greater than the other pulse.

17. The method of claim 16, wherein one pulse has a pressure amplitude at least twice the other pulse.

18. The method of claim 16, wherein one pulse is below the nanobubble pressure threshold for inertial cavitation followed by one above the threshold pressure threshold for inertial cavitation.

19. The method of claim 1, wherein the ultrasound energy is provided by non-focused ultrasound transducer.

20. The method of claim 1, wherein the targeted cell comprises wide-spread cancer micrometastasis in the subject.

21. The method of claim 1, wherein the cells comprise prokaryotic cells of microorganisms.

22. The method of any of claims 1 to 21, wherein the nanobubbles further comprise at least one therapeutic agent that is contained within the membrane or conjugated to the membrane of each nanobubble.

23. The method of claim 22, wherein the therapeutic agent further comprises at least one chemotherapeutic agent, anti-proliferative agent, biocidal agent, biostatic agent, or anti-microbial agent.

24. A method of treating cancer in a subject in need thereof, the method:
administering to the subject a plurality of nanobubbles, each nanobubble having a membrane that defines at least one internal void, which includes at least one gas, and a targeting moiety that is linked to an external surface of the membrane, wherein the target moiety binds to a cell surface molecule of a target cancer cell and the nanobubble has a size and/or diameter that facilitates internalization of the nanobubbles by the target cancer cell upon binding of the targeting moiety to the cell surface molecule; and
insonating the nanobubbles internalized into the target cancer cell with ultrasound energy effective to promote inertial cavitation of the internalized nanobubbles and apoptosis and/or necrosis of the target cancer cell.

25. The method of claim 24, wherein the nanobubbles have an average diameter of about 50 nm to about 400 nm.

26. The method of claim 24, wherein the targeting moiety is selected from the group consisting of polypeptides, polynucleotides, small molecules, elemental compounds, antibodies, and antibody fragments.

27. The method of claim 24, wherein the cancer cell surface molecule is a cancer cell antigen on the surface of a cancer cell.

28. The method of claim 27, wherein the cancer cell antigen comprises at least one of 5T4, α2β1 integrin, AXL receptor tyrosine kinase (AXL), B-cell maturation antigen (BCMA), c-MET (Hepatocyte Growth Factor Receptor), C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, collagen receptor, Cripto protein, CS1, delta-like canonical Notch ligand 3 (DLL3), endothelin receptor type B (EDNRB), ephrin A4 (EFNA4), epidermal growth factor receptor (EGFR), EGFRvIll, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), glycoprotein non-metastatic B (GPNMB), guanylate cyclase 2 C (GUCY2C), human epidermal growth factor receptor 2 (HER2), human epidermal growth factor receptor 3 (HERS), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), prostate-specific membrane antigen (PSMA), protein tyrosine kinase 7 (PTK7), protein tyrosine phosphatase mu (PTPmu) solute carrier family 44 member 4 (SLC44A4), SLIT like family member 6 (SLITRK6), STEAP family member 1 (STEAP1), tissue factor (TF), T cell immunoglobulin and mucin protein-1 (TIM-1), or trophoblast cell-surface antigen (TROP-2).

29. The method of claim 24, wherein the targeted cell is a prostate cancer cell and the cell surface molecule is PSMA.

30. The method of claim 24, wherein the membrane is a lipid membrane.

31. The method of claim 30, wherein the lipid membrane further includes at least one of glycerol, propylene glycol, pluronic (poloxamer), alcohols or cholesterols, that change the modulus and/or interfacial tension of the bubble membrane.

32. The method of claim 30, wherein the nanobubbles have a lipid concentration of at least about 5 mg/ml.

33. The method of claim 32, wherein the lipid membrane includes a mixture of at least two of dipalmitoylphosphatidylcholine (DPPC), dibehenoylglycerophosphocoline (DBPC), distearoylphosphatidylcholine (DSPC), diarachidonylphosphatidylcholine (DAPC), dioleoylphosphatidylethanolamine (DOPE), dipalmitoylphosphatidylethanolamine (DPPE), and distearoylphosphatidylethanolamine (DSPE); dipalmitoylphosphatidic acid (DPPA) or PEG functionalized lipids thereof.

34. The method of claim 33, wherein the mixture of lipids includes at least about 50% by weight of dibehenoylglycerophosphocoline (DBPC) and less than about 50% by weight of a combination of additional phospholipids selected from the group consisting of dipalmitoylphosphatidylcholine (DPPC), distearoylphosphatidylcholine (DSPC), diarachidonylphosphatidylcholine (DAPC), dioleoylphosphatidylethanolamine (DOPE), dipalmitoylphosphatidylethanolamine (DPPE), distearoylphosphatidylethanolamine (DSPE), dipalmitoylphosphatidic acid (DPPA), or PEG functionalized phospholipids thereof.

35. The method of claim 24, wherein gas comprises a perfluorocarbon gas.

36. The method of claim 24, wherein the insonation induces cell death without adversely effecting normal cells and tissues.

37. The method of claim 24, wherein the insonation is at a duty cycle of about 1% to about 50%, an ultrasound frequency of about 1 MHz to about 12 MHz, an intensity of about 0.1 W/cm² to about 3 W/cm², a pressure amplitude of about 50 kPa to about 1 MPa, and a time of about 1 minute to about 10 minutes.

38. The method of claim 24, wherein the insonation comprises two ultrasound pulse sequences with pulses of different pressure amplitudes sent to tissue in which the nanobubbles are administered, wherein one pulse has a pressure amplitude greater than the other pulse.

39. The method of claim 38, wherein one pulse has a pressure amplitude at least twice the other pulse.

40. The method of claim 38, wherein one pulse is below the nanobubble pressure threshold for inertial cavitation followed by one above the threshold pressure threshold for inertial cavitation.

41. The method of claim 24, wherein the ultrasound energy is provided by non-focused ultrasound transducer.

42. The method of claim 24, wherein the targeted cell comprises wide-spread cancer micrometastasis in the subject.

43. The method of claim 24, wherein the cells comprise prokaryotic cells of microorganisms.

44. The method of any of claims 24 to 43, wherein the nanobubbles further comprise at least one therapeutic agent that is contained within the membrane or conjugated to the membrane of each nanobubble.

45. The method of claim 44, wherein the therapeutic agent further comprises at least one chemotherapeutic agent, anti-proliferative agent, biocidal agent, biostatic agent, or anti-microbial agent.

46. A system for treating cancer in a subject, the system comprising:
an ultrasound source configured to non-invasively deliver ultrasound energy to cancer cells in the subject;
a plurality of nanobubbles, each nanobubble having a membrane that defines at least one internal void, which includes at least one gas, and a targeting moiety that is linked to an external surface of the membrane, wherein the target moiety binds to a cell surface molecule of a target cancer cell and the nanobubble has a size and/or diameter that facilitates internalization of the nanobubbles by the target cancer cell upon binding of the targeting moiety to the cell surface molecule; and
a controller coupled to the ultrasound source configured to cause insonation of cancer cells during an insonation time and promote inertial cavitation of nanobubbles internalized by the cancer cells.

47. The system of claim 46, wherein the insonation is at a duty cycle of about 1% to about 50%, an ultrasound frequency of about 1 MHz to about 12 MHz, an intensity of about 0.1 W/cm2 to about 3 W/cm2, a pressure amplitude of about 50 kPa to about 1 MPa, and a time of about 1 minute to about 10 minutes.

48. The system of claim 46, wherein the insonation comprises two ultrasound pulse sequences with pulses of different pressure amplitudes sent to tissue in which the nanobubbles are administered, wherein one pulse has a pressure amplitude greater than the other pulse.

49. The system of claim 48, wherein one pulse has a pressure amplitude at least twice the other pulse.

50. The system of claim 48, wherein one pulse is below the nanobubble pressure threshold for inertial cavitation followed by one above the threshold pressure threshold for inertial cavitation.

51. The system of claim 46, wherein the ultrasound energy is provided by non-focused ultrasound transducer.
